(19) United States
(12) Patent Application Publication
(10) Pub. No.: US 2015/0307596 A1
(43) Pub. Date: Oct. 29, 2015

(54) USE OF AN ION EXCHANGE MEMBRANE TO REMOVE IMPURITIES FROM CELL-BINDING AGENT CYTOTOXIC AGENT CONJUGATES

(71) Applicant: IMMUNOGEN, INC., Waltham, MA (US)

(72) Inventors: Xinfang Li, Newton, MA (US); Wenjie CHENG, Lexington, MA (US)

(21) Appl. No.: 14/430,744

(22) PCT Filed: Oct. 4, 2013
(86) PCT No.: PCT/US13/63503

§ 371 Date: Mar. 24, 2015

Related U.S. Application Data
(60) Provisional application No. 61/709,871, filed on Oct. 4, 2012.

(51) Int. Cl. C07K 16/06; C07K 16/40; A61K 47/48; C07K 16/28

(52) U.S. Cl. CPC C07K 16/065; C07K 16/2896; C07K 16/2863; C07K 16/2878; C07K 16/40; A61K 47/48561; C07K 2317/24

(57) ABSTRACT

The invention provides processes for preparing purified cell-binding agent cytotoxic agent conjugates comprising subjecting a mixture comprising a cell-binding agent cytotoxic agent conjugate and one or more impurities to an ion exchange chromatography membrane to remove at least a portion of the impurities from the mixture, thereby providing a purified cell-binding agent cytotoxic agent conjugate.

### CROSS-REFERENCE TO RELATED APPLICATIONS

This patent application claims the benefit of U.S. Provisional Patent Application No. 61/709,871, filed Oct. 4, 2012, which is incorporated by reference

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 8,279 Byte ASCII (Text) file named “714288SequenceListing.TXT,” created on Oct. 3, 2013.

### BACKGROUND OF THE INVENTION

Antibody-drug conjugates which are useful for the treatment of cancer and other diseases are commonly composed of three distinct elements: a cell-binding agent; a linker; and a cytotoxic agent. One of the commonly used manufacturing processes comprises a modification step, in which the cell-binding agent is reacted with a bifunctional linker to form a cell-binding agent covalently attached to a linker having a reactive group, a purification step in which the modified antibody is purified from the other components of the modification reaction, a conjugation step, in which the modified cell-binding agent is reacted with a cytotoxic agent to form a covalent chemical bond from the linker (using the reactive group) to the cytotoxic agent, and a second purification step, in which the conjugate is purified from the other components of the conjugation reaction.

Recent clinical trials have shown a promising role for antibody-drug conjugates in the treatment of many different types of cancers. Therefore, there is a need to produce conjugates of high purity and high stability that can be used to treat patients. Despite advances in preparing antibody-drug conjugates, current processes are limited by several factors. For example, the conjugates produced by these processes comprise an increased amount of impurities, including free cytotoxic agent (e.g., cytotoxic agent dimer related species) and/or high molecular weight species (e.g., dimers and other higher order aggregates). Current purification methods employed in the art, such as tangential flow filtration and adsorptive chromatography, do not efficiently remove these impurities without significantly decreasing the yield and/or are cumbersome for large scale manufacturing processes.

Thus, there remains a need for improved processes of preparing antibody-drug conjugates that are more stable and are of higher purity than antibody-drug conjugates produced by current processes. The invention provides such a process. These and other advantages of the invention, as well as additional inventive features, will be apparent from the description of the invention provided herein.

### BRIEF SUMMARY OF THE INVENTION

The invention provides processes for preparing purified cell-binding agent cytotoxic agent conjugates comprising subjecting a mixture comprising a cell-binding agent cytotoxic agent conjugate and one or more impurities to an ion exchange chromatography membrane to remove at least a portion of the impurities from the mixture, thereby providing a purified cell-binding agent cytotoxic agent conjugate. The present invention also includes a conjugate comprising a cell-binding agent chemically coupled to a cytotoxic agent prepared according to the processes described herein.

### DETAILED DESCRIPTION OF THE INVENTION

One of ordinary skill in the art will appreciate that conjugates comprising a cell-binding agent, such as an antibody, chemically coupled to a cytotoxic agent (“antibody-cytotoxic agent conjugates”) typically are prepared by modifying an antibody with a bifunctional crosslinking reagent at a low pH (i.e., pH 7.0 or below), purifying the antibody having linkers bound thereto, conjugating a cytotoxic agent to the antibody having linkers bound thereto, and purifying the antibody-cytotoxic agent conjugate. Recently, methods have been developed to produce conjugates of increased stability by maximizing the amount of linker stably bound to the cell-binding agent and minimizing undesirable side reactions that lead to conjugate instability. For example, methods have been developed in which the process for making the conjugate is performed in one step (see. e.g., the processes described in U.S. Patent Application Publication No. 20120253021) and/or at a high pH (e.g., a pH of 7 or higher) (see, e.g., the processes described International Patent Application Publication No. WO 2012135522) in order to increase the level of desirable species of cell-binding agents having a linker stably bound thereto and reduce the level of undesirable reaction products (e.g., cell-binding agents having a linker unstably bound thereto). Although such processes produce conjugates of increased stability, it has been discovered that these processes result in conjugates having an increased levels of impurities, such as free cytotoxic agent (e.g., cytotoxic agent dimer related species) and/or high molecular weight species (e.g., dimers and other higher order aggregates). Current purification methods employed in the art, such as tangential flow filtration and adsorptive chromatography, do not efficiently remove these impurities without significantly decreasing the yield and/or are cumbersome for large scale manufacturing process.

It was surprisingly discovered that an ion exchange chromatography membrane can be used to remove at least a portion of the impurities from a mixture comprising a cell-binding agent cytotoxic agent conjugate. In particular, it was unexpectedly discovered that free cytotoxic agent (e.g., cytotoxic agent dimer related species) can be effectively and efficiently removed from a mixture comprising a cell-binding agent cytotoxic agent conjugate by subjecting the mixture to an ion exchange chromatography membrane. Accordingly, the invention provides processes for manufacturing cell-binding agent-cytotoxic agent conjugates of increased purity and stability comprising subjecting a mixture comprising a cell-binding agent cytotoxic agent conjugate and one or more impurities to an ion exchange chromatography membrane.

The invention provides a process for preparing a purified cell-binding agent cytotoxic agent conjugate comprising subjecting a mixture comprising a cell-binding agent cytotoxic agent conjugate and one or more impurities to an ion exchange chromatography membrane to remove at least a portion of the impurities from the mixture, thereby providing a purified cell-binding agent cytotoxic agent conjugate. The ion exchange chromatography membrane can be used to remove a variety of impurities commonly found in a mixture comprising a cell-binding agent cytotoxic agent conjugate. For example, the ion exchange chromatography membrane can be used to remove one or more impurities selected from the group of cytotoxic agent dimers, aggregates of the cell-binding agent cytotoxic agent conjugate, free cytotoxic agent, unconjugated linker, and mixtures thereof.

In one embodiment, the mixture comprising a cell-binding agent cytotoxic agent conjugate comprises cytotoxic agent dimers as an impurity, and the ion exchange chromatography membrane removes some portion of the cytotoxic agent dimers from the mixture to provide the purified cell-binding agent cytotoxic agent conjugate. In another embodiment, the mixture comprising a cell-binding agent cytotoxic agent conjugate comprises aggregates of the cell-binding agent cytotoxic agent conjugate as an impurity, and the ion exchange chromatography membrane removes some portion of the aggregates of the cell-binding agent cytotoxic agent from the mixture to provide the purified cell-binding agent cytotoxic agent conjugate. In another embodiment, the mixture comprising a cell-binding agent cytotoxic agent conjugate comprises free cytotoxic agent as an impurity, and the ion exchange chromatography membrane removes some portion of the free cytotoxic agent from the mixture to provide the purified cell-binding agent cytotoxic agent conjugate. In another embodiment, the mixture comprising a cell-binding agent cytotoxic agent conjugate comprises unconjugated linker as an impurity, and the ion exchange chromatography membrane removes some portion of the unconjugated linker from the mixture to provide the purified cell-binding agent cytotoxic agent conjugate.

In a preferred embodiment, the mixture comprising a cell-binding agent cytotoxic agent conjugate comprises cytotoxic agent dimers chemically coupled to each other through a linker (e.g., DM1-MCC-DM1; DM1-SPP-DM1; or DM1-CX1-1-DM1) as an impurity, and the ion exchange chromatography membrane removes some portion of the cytotoxic agent dimers chemically coupled to each other through a linker (e.g., DM1-MCC-DM1; DM1-SPP-DM1; or DM1-CX1-1-DM1) from the mixture to provide the purified cell-binding agent cytotoxic agent conjugate. In another preferred embodiment, the mixture comprising a cell-binding agent cytotoxic agent conjugate comprises cytotoxic agent dimers that are not chemically coupled to each other through a linker (e.g., DM1-DM1) as an impurity, and the ion exchange chromatography membrane removes some portion of the cytotoxic agent dimers that are not chemically coupled to each other through a linker (e.g., DM1-DM1) from the mixture to provide the purified cell-binding agent cytotoxic agent conjugate.

When the mixture comprising a cell-binding agent cytotoxic agent conjugate and one or more impurities is subjected to an ion exchange chromatography membrane, the resulting purified cell-binding agent cytotoxic agent conjugate comprises a reduced level of at least one or more impurities as compared to the level of the one or more impurities in the mixture prior to subjecting the mixture to the ion exchange chromatography membrane. For example, the ion exchange chromatography membrane removes at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or even 100% of the one or more impurities in the mixture as compared to the level of the one or more impurities in the mixture prior to subjecting the mixture to the ion exchange chromatography membrane. In one embodiment, the ion exchange chromatography membrane removes about 10% to about 100%, about 10% to about 90%, about 20% to about 100%, about 20% to about 90%, about 20% to about 80%, about 30% to about 100%, about 30% to about 90%, about 30% to about 80%, about 40% to about 80%, about 40% to about 90%, about 40% to about 100%, about 50% to about 80%, about 50% to about 90%, about 50% to about 100% (e.g., about 60% to about 90%, about 70% to about 90%), about 60% to about 100%, about 70% to about 100%, about 80% to about 100%, about 90% to about 100%, or about 95% to about 100% (e.g., about 96% to about 100%, about 97% to about 100%, about 98% to about 100%, about 99% to about 100%, about 95% to about 96%, about 95% to about 97%, about 95% to about 98%, or about 95% to about 99%) of the one or more impurities in the mixture as compared to the level of the one or more impurities in the mixture prior to subjecting the mixture to the ion exchange chromatography membrane.

In one embodiment, the pH of the mixture comprising the cell-binding agent cytotoxic agent conjugate and one or more impurities is adjusted prior to subjecting the mixture to an ion exchange chromatography membrane. The pH of the mixture comprising the cell-binding agent cytotoxic agent conjugate and one or more impurities preferably is about 4 to about 9 (e.g., a pH of about 4.5 to about 8.5, about 5 to about 8, about 5.5 to about 7.5, about 6 to about 7, about 6.5 to about 7.5, about 7 to about 8, about 8 to about 9, about 4.5 to about 6, or about 4.5 to about 5). In some embodiments, the pH of the mixture is about 6 to about 6.5 (e.g., a pH of 5.5 to 7, a pH of 5.7 to 6.8, a pH of 5.8 to 6.7, a pH of 5.9 to 6.6, or a pH of 6 to 6.5), a pH of about 6 or below (e.g., a pH of about 4 to 6, about 4 to about 5.5, about 4 to about 4.5, about 4 to about 5, about 5 to 6), or a pH of about 6.5 or greater (e.g., a pH of 6.5 to about 9, about 6.5 to about 7, about 7 to about 9, about 7.5 to about 9, or 6.5 to about 8). In one embodiment, the pH of the mixture is greater than 7.5 (e.g., a pH of 7.6 to about 9, 7.7 to about 9, about 7.8 to about 9, about 7.9 to about 9, 7.6 to about 8.5, 7.6 to about 8, 7.7 to about 8.5, 7.7 to about 8, about 7.8 to about 8.4, about 7.8 to about 8.2, about 8 to about 9, or about 8 to about 8.5). For example, the pH of the mixture can be a pH of 7.6, 7.7, 7.8, 7.9, 8, 8.1, 8.2, 8.3, 8.4, 8.5, 8.6, 8.7, 8.8, 8.9, or 9. In another embodiment, the pH of the mixture is about 4.8 (e.g., about 4.5 to about 5, about 4.6 to about 5, or about 4.7 to about 4.9).

A variety of ion exchange chromatography membranes are known in the art and can be used in accordance with the invention described herein. In one embodiment, the ion exchange chromatography membrane is an anion exchange membrane, such as a Q membrane. In another embodiment, the ion exchange chromatography membrane is a cation exchange membrane, such as an S membrane. In one embodiment, the ion exchange chromatography membrane is an endotoxin removal exchange membrane. Q, S, and Endotoxin (E) membranes are commercially available, for example, from Pall Corporation and Sartorius Stedim Biotech.

In a preferred embodiment, the ion exchange chromatography membrane is an anion exchange membrane (e.g., a Q membrane). An anion exchange membrane is a positively charged microporous membrane. In one embodiment, the positively charged anion exchange moiety is quaternary ammonium group. In one embodiment, the positively charged microporous membrane comprises a porous substrate and a crosslinked coating having pendant cationic groups (see for example, those described in U.S. Pat. Nos. 6,780,327, 6,851,561, 7,094,347, 7,223,341, 7,396,465). In one embodiment, the porous substrate is hydrophilic (e.g., polyethersulfone or cross-linked cellulose matrix). In another embodiment, the cationic group is quaternary ammonium group. In another embodiment, the anion exchange membrane is a positively charged microporous membrane comprising a porous polyethersulfone substrate and a crosslinked coating having pendant quaternary ammonium groups.

A number of processes for preparing cell-binding agent-cytotoxic agent conjugates have been described (see, e.g., U.S. Patent Application Publication No. 20120253021; International Patent Application Publication No. WO 2012135522; U.S. Pat. No. 5,208,020; U.S. Pat. No. 6,441,163; U.S. Pat. No. 7,811,572; U.S. Patent Application Publication No. 20060182750; U.S. Patent Application Publication No. 20080145374; and U.S. Patent Application Publication No. 20110003969).

In one embodiment, the invention provides a process for preparing a conjugate comprising a cell-binding agent chemically coupled to a cytotoxic agent, wherein the modification reaction and the conjugation reaction are combined into a single step, followed by a purification step (i.e., the one-step process described in U.S. Patent Application Publication No. 20120253021), and wherein the process comprises subjecting the mixture comprising a cell-binding agent cytotoxic agent conjugate and one or more impurities to an ion exchange chromatography membrane either before or after the purification step. The one-step process comprises contacting a cell-binding agent (e.g., an antibody) with a cytotoxic agent to form a first mixture comprising the cell-binding agent and the cytotoxic agent, and then contacting the first mixture comprising the cell-binding agent and the cytotoxic agent with a bifunctional crosslinking reagent comprising a linker, in a solution having a pH of about 4 to about 9 to provide a second mixture comprising the cell-binding agent cytotoxic agent conjugate and one or more impurities (e.g., free cytotoxic agent and reaction by-products), wherein the cell-binding agent is chemically coupled through the linker to the cytotoxic agent. The second mixture is then subjected to purification to provide a purified cell-binding agent cytotoxic agent conjugate. The second mixture comprising a cell-binding agent cytotoxic agent conjugate and one or more impurities is subjected to an ion exchange chromatography membrane before the purification step, after the purification step, or both.

The one-step reaction preferably is performed at a pH of about 4 to about pH 9 (e.g., a pH of about 4.5 to about 8.5, about 5 to about 8, about 5.5 to about 7.5, about 6 to about 7, about 6 to about 8, about 6 to about 9, or about 6.5 to about 7.5). In some embodiments, the reaction is performed at a pH of about 6 to about 8 (e.g., a pH of about 6, about 6.5, about 7, about 7.5, or about 8).

In one embodiment, the reaction is performed at a pH of about 7.1, about 7.2, about 7.3, about 7.4, about 7.5, about 7.6, about 7.7, about 7.8, about 7.9, about 8, about 8.1, about 8.2, about 8.3, about 8.4, about 8.5, about 8.6, about 8.7, about 8.8, about 8.9, or about 9. In another embodiment, the reaction is performed at a pH of about 7.5 to about 9, about 7.5 to about 8.5, about 7.5 to about 8, about 7.8 to about 9, about 7.8 to about 8.5, about 7.8 to about 8, about 8 to about 9, about 8 to about 8.5, or about 8.5 to about 9. In another embodiment the reaction is performed at a pH of about 7.8 (e.g., a pH of 7.6 to 8.0 or a pH of 7.7 to 7.9). In another embodiment, the modification reaction is performed at a pH of about 8 (e.g., a pH of 7.8 to 8.2 or a pH of 7.9 to 8.1).

In another embodiment, the reaction is performed at a pH that is greater than 7.5 (e.g., a pH of 7.6 to about 9, 7.7 to about 9, about 7.8 to about 9, about 7.9 to about 9, 7.6 to about 8.5, 7.6 to about 8, 7.7 to about 8.5, 7.7 to about 8, about 7.8 to about 8.4, about 7.8 to about 8.2, about 8 to about 9, or about 8 to about 8.5).

In one embodiment, the contacting is effected by providing the cell-binding agent, then contacting the cell-binding agent with the cytotoxic agent to form a first mixture comprising the cell-binding agent and the cytotoxic agent, and then contacting the first mixture comprising the cell-binding agent and the cytotoxic agent with the bifunctional crosslinking reagent. For example, in one embodiment, the cell-binding agent is provided in a reaction vessel, the cytotoxic agent is added to the reaction vessel (thereby contacting the cell-binding agent), and then the bifunctional crosslinking reagent is added to the mixture comprising the cell-binding agent and the cytotoxic agent (thereby contacting the mixture comprising the cell-binding agent and the cytotoxic agent). In one embodiment, the cell-binding agent is provided in a reaction vessel, and the cytotoxic agent is added to the reaction vessel immediately following providing the cell-binding agent to the vessel. In another embodiment, the cell-binding agent is provided in a reaction vessel, and the cytotoxic agent is added to the reaction vessel after a time interval following providing the cell-binding agent to the vessel (e.g., about 5 minutes, about 10 minutes, about 20 minutes, about 30 minutes, about 40 minutes, about 50 minutes, about 1 hour, about 1 day or longer after providing the cell-binding agent to the space). The cytotoxic agent can be added quickly (i.e., within a short time interval, such as about 5 minutes, about 10 minutes) or slowly (such as by using a pump).

The mixture comprising the cell-binding agent and the cytotoxic agent can be then contacted with the bifunctional crosslinking reagent either immediately after contacting the cell-binding agent with the cytotoxic agent or at some later point (e.g., about 5 minutes to about 8 hours or longer) after contacting the cell-binding agent with the cytotoxic agent. For example, in one embodiment, the bifunctional crosslinking reagent is added to the mixture comprising the cell-binding agent and the cytotoxic agent immediately after the addition of the cytotoxic agent to the reaction vessel comprising the cell-binding agent. Alternatively, the mixture comprising the cell-binding agent and the cytotoxic agent can be contacted with the bifunctional crosslinking reagent at about 5 minutes, about 10 minutes, about 20 minutes, about 30 minutes, about 1 hour, about 2 hours, about 3 hours, about 4 hours, about 5 hours, about 6 hours, about 7 hours, about 8 hours, or longer after contacting the cell-binding agent with the cytotoxic agent.

In another embodiment, the cytotoxic agent and the bifunctional agent are added through multiple cycles (e.g., 1, 2, 3, 4, 5 or more cycles). For example, the invention provides a process comprising the steps of: a) contacting a cell-binding agent with a cytotoxic agent to form a first mixture comprising the cell-binding agent and the cytotoxic agent; and then contacting the first mixture with a bifunctional crosslinking reagent comprising a linker, in a solution having a pH of about 4 to about 9 to provide a second mixture comprising the cell-binding agent cytotoxic agent conjugate and one or more impurities (e.g., free cytotoxic agent and reaction by-products), wherein the cell-binding agent is chemically coupled through the linker to the cytotoxic agent; b) contacting the second mixture with the cytotoxic agent to form a third mixture; and then contacting the third mixture with the bifunctional crosslinking reagent at a pH of about 4 to about 9 to provide a fourth mixture; and c) purifying the fourth mixture to provide the purified cell-binding agent cytotoxic agent conjugate. In one embodiment, step b) is carried out after a time interval (e.g., about 1 hour, about 2 hours, about 3 hours or longer) following step a). In another embodiment, step b) can be repeated several times (e.g., 1, 2, 3, 4 or more times) before step c) is carried out. The additional step b) can be carried out after a time interval (e.g., about 1 hour, about 2 hours, about 3 hours or longer) following the initial step b).

In another embodiment, the bifunctional crosslinking reagent is added before the complete addition of the cytotoxic agent. For example, in one embodiment, the cytotoxic agent is added to the cell-binding agent continuously over a time interval (e.g., over about 5 minutes, about 10 minutes, about 30 minutes, about 1 hour, about 2 hours, about 3 hours, or longer) to form a mixture comprising the cell-binding agent and the cytotoxic agent. Before the addition of the cytotoxic agent is complete, the bifunctional crosslinking reagent is added to the mixture comprising the cell-binding agent and the cytotoxic agent, provided that at any time, the cytotoxic agent is in molar excess of the bifunctional crosslinking reagent. In one embodiment, the bifunctional crosslinking reagent is added continuously over a time interval (e.g., over about 5 minutes, about 10 minutes, about 30 minutes, about 1 hour, about 2 hours, about 3 hours, or longer).

After the mixture comprising the cell-binding agent and the cytotoxic agent is contacted with the bifunctional crosslinking reagent, the reaction is allowed to proceed for about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, or longer (e.g., about 30 hours, about 35 hours, about 40 hours, about 45 hours, or about 48 hrs).

Thus, in one embodiment, the invention provides a process for preparing a purified cell-binding agent cytotoxic agent conjugate comprising contacting a cell-binding agent with a cytotoxic agent to form a first mixture comprising the cell-binding agent and the cytotoxic agent, then contacting the first mixture with a bifunctional crosslinking reagent comprising a linker, in a solution having a pH of about 4 to about 9, to provide a second mixture comprising the cell-binding agent cytotoxic agent conjugate comprising the cell-binding agent chemically coupled through the linker to the cytotoxic agent and one or more impurities; (b) subjecting the second mixture to an ion exchange chromatography membrane to remove at least a portion of the impurities, thereby providing a purified second mixture of the cell-binding agent cytotoxic agent conjugate; and (c) subjecting the purified second mixture after step (b) to tangential flow filtration, selective precipitation, non-adsorptive chromatography, adsorptive filtration, adsorptive chromatography, or a combination thereof, to further purify the cell-binding agent-cytotoxic agent conjugate from the impurities and thereby prepare a purified third mixture of the cell-binding agent-cytotoxic agent conjugate, wherein the purified third mixture comprises a reduced amount of the impurities as compared to the purified second mixture. Any purification method described herein can be used in the inventive process. In a preferred embodiment, tangential flow filtration, adsorptive chromatography, or non-adsorptive chromatography is utilized as the purification step.

In one embodiment of the invention, contacting a cell-binding agent with a bifunctional crosslinking reagent (i.e., the modification reaction) produces a first mixture comprising the cell-binding agent having linkers bound thereto and one or more impurities (e.g., reactants and other by-products). In some embodiments of the invention, the first mixture comprises the cell-binding agent having linkers stably and unstably bound thereto and one or more impurities (e.g., reactants and other by-products). A linker is “stably” bound to the cell-binding agent when the covalent bond between the linker and the cell-binding agent is not substantially weakened or severed under normal storage conditions over a period of time, which could range from a few months to a few years. In contrast, a linker is “unstably” bound to the cell-binding agent when the covalent bond between the linker and the cell-binding agent is substantially weakened or severed under normal storage conditions over a period of time, which could range from a few months to a few years.

The modification reaction preferably is performed at a pH of about 4 to about pH 9 (e.g., a pH of about 4.5 to about 8.5, about 5 to about 8, about 5.5 to about 7.5, about 6 to about 7, about 6 to about 8, about 6 to about 9, or about 6.5 to about 7.5). In some embodiments, the modification reaction is performed at a pH of about 6 to about 8 (e.g., a pH of about 6, about 6.5, about 7, about 7.5, or about 8).

In one embodiment, the modification reaction is performed at a pH of about 7.1, about 7.2, about 7.3, about 7.4, about 7.5, about 7.6, about 7.7, about 7.8, about 7.9, about 8, about 8.1, about 8.2, about 8.3, about 8.4, about 8.5, about 8.6, about 8.7, about 8.8, about 8.9, or about 9. In another embodiment, the modification reaction is performed at a pH of about 7.5 to about 9, about 7.5 to about 8.5, about 7.5 to about 8, about 7.8 to about 9, about 7.8 to about 8.5, about 7.8 to about 8, about 8 to about 9, about 8 to about 8.5, or about 8.5 to about 9. In another embodiment the modification reaction is performed at a pH of about 7.8 (e.g., a pH of 7.6 to 8.0 or a pH of 7.7 to 7.9). In another embodiment, the modification reaction is performed at a pH of about 8 (e.g., a pH of 7.8 to 8.2 or a pH of 7.9 to 8.1).

In another embodiment, the modification reaction is performed at a pH that is greater than 7.5 (e.g., a pH of 7.6 to about 9, 7.7 to about 9, about 7.8 to about 9, about 7.9 to about 9, 7.6 to about 8.5, 7.6 to about 8, 7.7 to about 8.5, 7.7 to about 8, about 7.8 to about 8.4, about 7.8 to about 8.2, about 8 to about 9, or about 8 to about 8.5). For example, the inventive process comprises contacting a cell-binding agent with a bifunctional crosslinking reagent in a solution having a pH of 7.6, 7.7, 7.8, 7.9, 8, 8.1, 8.2, 8.3, 8.4, 8.5, 8.6, 8.7, 8.8, 8.9, or 9.

In one embodiment of the invention, purification of the modified cell-binding agent from impurities produced during the modification reaction (e.g., reactants and by-products) is carried out by subjecting the mixture produced by the modification reaction (i.e., the first mixture) to a purification process. In this regard, the first mixture can be purified using tangential flow filtration (TFF), e.g., a membrane-based tangential flow filtration process, non-adsorptive chromatography, adsorptive chromatography, adsorptive filtration, or selective precipitation, or any other suitable purification process, as well as combinations thereof. This first purification step provides a purified first mixture, i.e., an increased concentration of the cell-binding agents having linkers bound thereto and a decreased amount of unbound bifunctional crosslinking reagent, as compared to the first mixture prior to purification in accordance with the invention. Preferably, the first mixture is purified using tangential flow filtration or adsorptive chromatography (e.g., ion exchange chromatography, such as ceramic hydroxyapatite).

After purification of the first mixture to obtain a purified first mixture of cell-binding agents having linkers bound thereto, a cytotoxic agent is conjugated to the cell-binding agents having linkers bound thereto in the first purified mixture by reacting the cell-binding agents having linkers bound thereto with a cytotoxic agent in a solution having a pH from about 4 to about 9 to form a second mixture, wherein a second mixture comprising the cell-binding agent chemically coupled through the linker to the cytotoxic agent and one or more impurities (e.g., free cytotoxic agent and reaction by-products) is produced.

Optionally, purification of the modified cell-binding agent may be omitted. Thus, in one embodiment of the invention, the first mixture comprising the cell-binding agent having linkers bound thereto, as well as reactants and other by-products, is not subjected to a purification process. In such a situation, the cytotoxic agent may be added simultaneously with the crosslinking reagent or at some later point, e.g., 1, 2, 3, or more hours after addition of the crosslinking reagent to the cell-binding agent. The modified cell-binding agent is conjugated to a cytotoxic agent (e.g., a maytansinoid) by reacting the modified cell-binding agent with the cytotoxic agent in a solution having a pH from about 4 to about 9, wherein the conjugation step results in formation of a mixture of stable cell-binding agent-cytotoxic agent conjugates, non-stable cell-binding agent-cytotoxic agent conjugates, non-conjugated cytotoxic agent (i.e., “free” cytotoxic agent), reactants, and by-products.

The conjugation reaction preferably is performed at a pH of about 4 to about pH 9 (e.g., a pH of about 4.5 to about 8.5, about 5 to about 8, about 5.5 to about 7.5, about 6.0 to about 7, or about 6.5 to about 7.5). In some embodiments, the conjugation reaction is performed at a pH of about 6 to about 6.5 (e.g., a pH of 5.5 to 7, a pH of 5.7 to 6.8, a pH of 5.8 to 6.7, a pH of 5.9 to 6.6, or a pH of 6 to 6.5), a pH of about 6 or below (e.g., a pH of about 4 to 6, about 4 to about 5.5, about 5 to 6) or at a pH of about 6.5 or greater (e.g., a pH of 6.5 to about 9, about 6.5 to about 7, about 7 to about 9, about 7.5 to about 9, or 6.5 to about 8). In one embodiment, the conjugation reaction is performed at a pH of about 4 to a pH less than 6 or at a pH of greater than 6.5 to 9. When the conjugation step is performed at a pH of about 6.5 or greater, some sulfhydryl-containing cytotoxic agents may be prone to dimerize by disulfide-bond formation. In one embodiment, removal of trace metals and/or oxygen from the reaction mixture, as well as optional addition of antioxidants or the use of linkers with more reactive leaving groups, or addition of cytotoxic agent in more than one aliquot, may be required to allow for efficient reaction in such a situation.

Following the conjugation step, the mixture comprising the cell-binding agent cytotoxic agent conjugate and one or more impurities is subjected to a purification step. In this regard, the conjugation mixture can be purified using tangential flow filtration (TFF), e.g., a membrane-based tangential flow filtration process, non-adsorptive chromatography, adsorptive chromatography, adsorptive filtration, or selective precipitation, or any other suitable purification process, as well as combinations thereof. One of ordinary skill in the art will appreciate that purification after the conjugation step enables the isolation of a purified conjugate comprising the cell-binding agent chemically coupled to the cytotoxic agent, wherein the conjugate has a reduced amount of impurities as compared to the conjugate prior to the purification step. In one embodiment, the mixture comprising the cell-binding agent cytotoxic agent conjugate and one or more impurities is subjected to an ion exchange chromatography membrane after the conjugation step and prior to the purification step in order to remove at least a portion of the impurities from the mixture prior to purification. In another embodiment, the mixture comprising the cell-binding agent cytotoxic agent conjugate and one or more impurities is subjected to an ion exchange chromatography membrane after the purification step in order to remove at least a portion of the impurities remaining in the mixture after purification.

Thus, in one embodiment, the invention provides a process for preparing a conjugate comprising a cell-binding agent chemically coupled to a cytotoxic agent, which process comprises a first purification step after the modification step and a second purification step after the conjugation step, wherein the process comprises subjecting the mixture comprising a cell-binding agent cytotoxic agent conjugate and one or more impurities to an ion exchange chromatography membrane either before or after the second purification step to remove at least a portion of the impurities from the mixture. In one embodiment, the invention provides a process for preparing a purified cell-binding agent cytotoxic agent conjugate comprising contacting a cell-binding agent with a bifunctional crosslinking reagent to covalently attach a linker to the cell-binding agent and thereby prepare a first mixture comprising cell-binding agents having linkers bound thereto, (b) subjecting the first mixture to tangential flow filtration, selective precipitation, non-adsorptive chromatography, adsorptive filtration, adsorptive chromatography, or a combination thereof and thereby prepare a purified first mixture of cell-binding agents having linkers bound thereto, (c) conjugating a cytotoxic agent to the cell-binding agents having linkers bound thereto in the purified first mixture by reacting the cell-binding agents having linkers bound thereto with a cytotoxic agent in a solution having a pH of about 4 to about 9 to prepare a second mixture comprising the cell-binding agent-cytotoxic agent conjugate comprising the cell-binding agent chemically coupled to the cytotoxic agent through the linker and one or more impurities, (d) subjecting the second mixture to an ion exchange chromatography membrane to remove at least a portion of the impurities, thereby providing a purified second mixture of the cell-binding agent cytotoxic agent conjugate; and (e) subjecting the purified second mixture after step (d) to tangential flow filtration, selective precipitation, non-adsorptive chromatography, adsorptive filtration, adsorptive chromatography, or a combination thereof, to further purify the cell-binding agent-cytotoxic agent conjugate from the impurities and thereby prepare a purified third mixture of the cell-binding agent-cytotoxic agent conjugate, wherein the purified third mixture comprises a reduced amount of the impurities as compared to the purified second mixture.

Any purification method described herein can be used in the inventive process. In one embodiment of the invention, tangential flow filtration (TFF, also known as cross flow filtration, ultrafiltration and diafiltration) and/or adsorptive chromatography resins are utilized in the purification steps. For example, the inventive process can comprise a first purification step using TFF after the modification step and a second purification step using TFF after the conjugation step. Alternatively, the inventive process can comprise a first purification step using adsorptive chromatography after the modification step and a second purification step using adsorptive chromatography after the conjugation step. The inventive process also can comprise a first purification step using adsorptive chromatography after the modification step and a second purification step using TFF after the conjugation step or a first purification step using TFF after the modification step and a second purification step using adsorptive chromatography after the conjugation step.

In one embodiment of the invention, non-adsorptive chromatography is utilized as the purification step. For example, the inventive process can comprise a first purification step using non-adsorptive chromatography after the modification step and a second purification step using non-adsorptive chromatography after the conjugation step.

In another embodiment, the invention provides a process for preparing a conjugate comprising a cell-binding agent chemically coupled to a cytotoxic agent, wherein the first mixture comprising cell-binding agents having linkers bound thereto is not subjected to purification following the modification reaction and prior to the conjugation reaction, and wherein the process comprises subjecting the mixture comprising a cell-binding agent cytotoxic agent conjugate and one or more impurities to an ion exchange chromatography membrane. When purification of the modified cell-binding agent is omitted, the invention provides a process for preparing a conjugate comprising a cell-binding agent chemically coupled to a cytotoxic agent, which process comprises a modification step, a conjugation step, and a first purification step after the conjugation step, wherein the process comprises subjecting the mixture comprising a cell-binding agent cytotoxic agent conjugate and one or more impurities to an ion exchange chromatography membrane either before or after the first purification step to remove at least a portion of the impurities from the mixture. In one embodiment, the invention provides process for preparing a purified cell-binding agent cytotoxic agent conjugate comprising contacting a cell-binding agent with a bifunctional crosslinking reagent to covalently attach a linker to the cell-binding agent and thereby prepare a first mixture comprising cell-binding agents having linkers bound thereto, (b) conjugating a cytotoxic agent to the cell-binding agents having linkers bound thereto in the first mixture by reacting the cell-binding agents having linkers bound thereto with a cytotoxic agent to prepare a second mixture comprising the cell-binding agent-cytotoxic agent conjugate comprising the cell-binding agent chemically coupled through the linker to the cytotoxic agent and one or more impurities, (c) subjecting the second mixture to an ion exchange chromatography membrane to remove at least a portion of the impurities, thereby providing a purified second mixture of the cell-binding agent cytotoxic agent conjugate; and (d) subjecting the purified second mixture after step (c) to tangential flow filtration, selective precipitation, non-adsorptive chromatography, adsorptive filtration, adsorptive chromatography, or a combination thereof, to further purify the cell-binding agent-cytotoxic agent conjugate from the impurities and thereby prepare a purified third mixture of the cell-binding agent-cytotoxic agent conjugate, wherein the purified third mixture comprises a reduced amount of the impurities as compared to the purified second mixture, and wherein the first mixture comprising the cell-binding agent having linkers bound thereto (as well as reactants and other by-products) prepared in step (a) is not subjected to a purification process prior to step (b). Any purification method described herein can be used as the purification step following the conjugation reaction. In a preferred embodiment, tangential flow filtration, adsorptive chromatography, or non-adsorptive chromatography is utilized as the purification step following the conjugation reaction.

In one embodiment, the invention provides a process for preparing a conjugate comprising a cell-binding agent chemically coupled to a cytotoxic agent, wherein the process comprises conjugating a pre-formed cytotoxic-agent-linker compound to a cell-binding agent, as described in U.S. Pat. No. 6,441,163 and U.S. Patent Application Publication Nos. 20110003969 and 20080145374, followed by a purification step, and wherein the process comprises subjecting the mixture comprising a cell-binding agent cytotoxic agent conjugate and one or more impurities to an ion exchange chromatography membrane either before or after the purification step. Any purification method described herein can be used in the inventive process. In a preferred embodiment, tangential flow filtration, adsorptive chromatography, or non-adsorptive chromatography is utilized as the purification step.

In one embodiment, the cytotoxic agent-linker compound is prepared by contacting a cytotoxic agent with a bifunctional crosslinking reagent comprising a linker to covalently attach the cytotoxic agent to the linker. The cytotoxic agent-linker compound optionally is subjected to purification before contacting cytotoxic agent-linker compound with the cell-binding agent.

In one embodiment of the invention, the inventive process described herein (e.g., the one-step process) comprises two separate purification steps following the conjugation step. When the inventive process comprises two separate purification steps following the conjugation step, the mixture comprising a cell-binding agent cytotoxic agent conjugate and one or more impurities can be subjected to an ion exchange chromatography membrane before either or both of the purification steps, or following the purification steps to remove at least a portion of the impurities from the mixture. Any purification method described herein can be used as the purification steps following the conjugation reaction. In a preferred embodiment, tangential flow filtration, adsorptive chromatography, non-adsorptive chromatography, or a combination thereof are utilized as the purification steps following the conjugation reaction.

In one embodiment, the mixture is subjected to purification prior to subjecting the mixture to an ion exchange chromatography membrane (e.g., a Q membrane or an S membrane). In one embodiment, the mixture is subjected to an ion exchange chromatography membrane prior to subjecting the mixture to purification. In yet another embodiment, the mixture is subject to an ion exchange chromatography membrane before and after subjecting the mixture to purification. In another embodiment, the mixture is subjected to purification membrane before and after subjection the mixture to an ion exchange chromatography membrane.

Any suitable TFF systems may be utilized for purification, including a Pellicon type system (Millipore, Billerica, Mass.), a Sartocon Cassette system (Sartorius AG, Edgewood, N.Y.), and a Centrasette type system (Pall Corp., East Hills, N.Y.).

Any suitable adsorptive chromatography resin may be utilized for purification. Preferred adsorptive chromatography resins include hydroxyapatite chromatography, hydrophobic charge induction chromatography (HCIC), hydrophobic interaction chromatography (HIC), ion exchange chromatography, mixed mode ion exchange chromatography, immobilized metal affinity chromatography (IMAC), dye ligand chromatography, affinity chromatography, reversed phase chromatography, and combinations thereof. Examples of suitable hydroxyapatite resins include ceramic hydroxyapatite (CHT Type I and Type II, Bio-Rad Laboratories, Hercules, Calif.), HA Ultrogel hydroxyapatite (Pall Corp., East Hills, N.Y.), and ceramic fluoroapatite (CFT Type I and Type II, Bio-Rad Laboratories, Hercules, Calif.). An example of a suitable HCIC resin is MEP Hypercel resin (Pall Corp., East Hills, N.Y.). Examples of suitable HIC resins include Butyl-Sepharose, Hexyl-Sepaharose, Phenyl-Sepharose, and Octyl Sepharose resins (all from GE Healthcare, Piscataway, N.J.), as well as Macro-prep Methyl and Macro-Prep t-Butyl resins (Biorad Laboratories, Hercules, Calif.). Examples of suitable ion exchange resins include SP-Sepharose, CM-Sepharose, and Q-Sepharose resins (all from GE Healthcare, Piscataway, N.J.), and Unosphere S resin (Bio-Rad Laboratories, Hercules, Calif.). Examples of suitable mixed mode ion exchangers include Bakerbond ABx resin (JT Baker, Phillipsburg N.J.). Examples of suitable IMAC resins include Chelating Sepharose resin (GE Healthcare, Piscataway, N.J.) and Profinity IMAC resin (Bio-Rad Laboratories, Hercules, Calif.). Examples of suitable dye ligand resins include Blue Sepharose resin (GE Healthcare, Piscataway, N.J.) and Affi-gel Blue resin (Bio-Rad Laboratories, Hercules, Calif.). Examples of suitable affinity resins include Protein A Sepharose resin (e.g., MabSelect, GE Healthcare, Piscataway, N.J.), where the cell-binding agent is an antibody, and lectin affinity resins, e.g. Lentil Lectin Sepharose resin (GE Healthcare, Piscataway, N.J.), where the cell-binding agent bears appropriate lectin binding sites. Alternatively an antibody specific to the cell-binding agent may be used. Such an antibody can be immobilized to, for instance, Sepharose 4 Fast Flow resin (GE Healthcare, Piscataway, N.J.). Examples of suitable reversed phase resins include C4, C8, and C18 resins (Grace Vydac, Hesperia, Calif.).

Any suitable non-adsorptive chromatography resin may be utilized for purification. Examples of suitable non-adsorptive chromatography resins include, but are not limited to, SEPHADEX™ G-25, G-50, G-100, SEPHACRYL™ resins (e.g., S-200 and S-300), SUPERDEX™ resins (e.g., SUPERDEX™ 75 and SUPERDEX™ 200), BIO-GEL® resins (e.g., P-6, P-10, P-30, P-60, and P-100), and others known to those of ordinary skill in the art.

The inventive process comprises performing the reactions described herein (e.g., the modification reaction, the conjugation reaction, or the one-step reaction) at any suitable temperature known in the art. For example, the reaction can occur at about 20° C. or less (e.g., about −10° C. (provided that the solution is prevented from freezing, e.g., by the presence of organic solvent used to dissolve the cytotoxic agent and the bifunctional crosslinking reagent) to about 20° C., about 0° C. to about 18° C., about 4° C. to about 16° C.), at room temperature (e.g., about 20° C. to about 30° C. or about 20° C. to about 25° C.), or at an elevated temperature (e.g., about 30° C. to about 37° C.). In one embodiment, the reaction occurs at a temperature of about 16° C. to about 24° C. (e.g., about 16° C., about 17° C., about 18° C., about 19° C., about 20° C., about 21° C., about 22° C., about 23° C., about 24° C., or about 25° C.).

In one embodiment, the inventive process described herein further comprises a quenching step to quench any unreacted cytotoxic agent and/or unreacted bifunctional crosslinking reagent. The quenching step is performed prior to purification of the cell-binding agent cytotoxic agent. Alternatively, the quenching step is performed after purification of the cell-biding agent cytotoxic agent. In one embodiment, the inventive process comprises (a) contacting a cell-binding agent with a cytotoxic agent to form a mixture comprising the cell-binding agent and the cytotoxic agent and then contacting the mixture comprising the cell-binding agent and the cytotoxic agent with a bifunctional crosslinking reagent comprising a linker, in a solution having a pH of about 4 to about 9 to provide a mixture comprising the cell-binding agent cytotoxic agent conjugate, wherein the cell-binding agent is chemically coupled through the linker to the cytotoxic agent, and impurities (e.g., free cytotoxic agent and reaction by-products), (b) subjecting the mixture comprising the cell-binding agent cytotoxic agent conjugate and one or more impurities to an ion exchange chromatography membrane, (c) quenching the mixture after step (b) to quench any unreacted cytotoxic agent and/or unreacted bifunctional crosslinking reagent, (d) subjecting the quenched mixture to an ion exchange chromatography membrane, (e) optionally holding the mixture, (f) optionally subjecting the mixture to an ion exchange chromatography membrane, and (g) purifying the mixture to provide a purified cell-binding agent cytotoxic agent conjugate. In another embodiment, the inventive process comprises (a) contacting a cell-binding agent with a cytotoxic agent to form a mixture comprising the cell-binding agent and the cytotoxic agent and then contacting the mixture comprising the cell-binding agent and the cytotoxic agent with a bifunctional crosslinking reagent comprising a linker, in a solution having a pH of about 4 to about 9 to provide a mixture comprising the cell-binding agent cytotoxic agent conjugate, wherein the cell-binding agent is chemically coupled through the linker to the cytotoxic agent, and impurities (e.g., free cytotoxic agent and reaction by-products), (b) subjecting the mixture comprising the cell-binding agent cytotoxic agent conjugate and one or more impurities to an ion exchange chromatography membrane, (c) optionally quenching the mixture after step (b) to quench any unreacted cytotoxic agent and/or unreacted bifunctional crosslinking reagent, (d) optionally subjecting the quenched mixture to an ion exchange chromatography membrane, (e) holding the mixture, (f) subjecting the mixture to an ion exchange chromatography membrane, and (g) purifying the mixture to provide a purified cell-binding agent cytotoxic agent conjugate.

In one embodiment, the mixture is quenched by contacting the mixture with a quenching reagent. As used herein, the “quenching reagent” refers to a reagent that reacts with the free cytotoxic agent and/or the bifunctional crosslinking reagent.

In one embodiment, maleimide or haloacetamide quenching reagents, such as 4-maleimidobutyric acid, 3-maleimidopropionic acid, N-ethylmaleimide, iodoacetamide, or iodoacetamidopropionic acid, can be used to ensure that any unreacted group (such as thiol) in the cytotoxic agent is quenched. The quenching step can help prevent the dimerization of the cytotoxic agent, particular the cytotoxic agent having an unreacted thiol group (such as DM1). The dimerized cytotoxic agent can be difficult to remove. The quenching step may also minimize any unwanted thiol-disulfide interchange reaction with the native antibody disulfide groups. Upon quenching with polar, charged thiol-quenching reagents (such as 4-maleimidobutyric acid or 3-maleimidopropionic acid), the excess, unreacted cytotoxic agent is converted into a polar, charged, water-soluble adduct that can be easily separated from the covalently-linked conjugate during the purification step. Quenching with non-polar and neutral thiol-quenching reagents can also be used.

In one embodiment, the mixture is quenched by contacting the mixture with a quenching reagent that reacts with the unreacted bifunctional crosslinking reagent. For example, nucleophiles can be added to the mixture in order to quench any unreacted bifunctional crosslinking reagent. The nucleophile preferably is an amino group containing nucleophile, such as lysine, taurine and hydroxylamine.

Alternatively, the mixture is quenched by lowering the pH of the mixture to about 5.0 (e.g., 4.8, 4.9, 5.0, 5.1 or 5.2). In another embodiment, the mixture is quenched by lowering the pH to less than 6.0, less than 5.5, less than 5.0, less than 4.8, less than 4.6, less than 4.4, less than 4.2, less than 4.0. Alternatively, the pH is lowered to about 4.0 (e.g., 3.8, 3.9, 4.0, 4.1 or 4.2) to about 6.0 (e.g., 5.8, 5.9, 6.0, 6.1 or 6.2), about 4.0 to about 5.0, about 4.5 (e.g., 4.3, 4.4, 4.5, 4.6 or 4.7) to about 5.0. In one embodiment, the mixture is quenched by lowering the pH of the mixture to 4.8.

In a preferred embodiment, the reaction (e.g., the modification step, the conjugation step, or the one-step reaction) is allowed to proceed to completion prior to contacting the mixture with a quenching reagent. In this regard, the quenching reagent is added to the mixture about 1 hour to about 48 hours (e.g., about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, or about 25 hours to about 48 hours) after the mixture comprising the cell-binding agent and the cytotoxic agent is contacted with the bifunctional crosslinking reagent.

The inventive process may optionally include the addition of sucrose to the reaction step (e.g., the modification step, the conjugation step, or the one-step reaction) to increase solubility and recovery of the cell-binding agent-cytotoxic agent conjugates. Desirably, sucrose is added at a concentration of about 0.1% (wv) to about 20% (wv) (e.g., about 0.1% (wv), 1% (wv), 5% (wv), 10% (wv), 15% (wv), or 20% (wv)). Preferably, sucrose is added at a concentration of about 1% (wv) to about 10% (wv) (e.g., about 0.5% (wv), about 1% (wv), about 1.5% (wv), about 2% (wv), about 3% (wv), about 4% (wv), about 5% (wv), about 6% (wv), about 7% (wv), about 8% (wv), about 9% (wv), about 10% (wv), or about 11% (wv)). In addition, the reaction step also can comprise the addition of a buffering agent. Any suitable buffering agent known in the art can be used. Suitable buffering agents include, for example, a citrate buffer, an acetate buffer, a succinate buffer, and a phosphate buffer. In one embodiment, the buffering agent is selected from the group consisting of HEPPSO (N-(2-hydroxyethyl)piperazine-N′-(2-hydroxypropanesulfonic acid)), POPSO (piperazine-1,4-bis-(2-hydroxy-propane-sulfonic acid) dehydrate), HEPES (4-(2-hydroxyethyl)piperazine-1-ethanesulfonic acid), HEPPS (EPPS) (4-(2-hydroxyethyl)piperazine-1-propanesulfonic acid), TES (N-[tris(hydroxymethyl)methyl]-2-aminoethanesulfonic acid), and a combination thereof.

In one embodiment, the inventive process further comprises one or more (e.g., one, two, or three) holding steps to release the unstably bound linkers from the cell-binding agent. The holding step comprises holding the mixture after modification of the cell-binding agent with a bifunctional crosslinking reagent, after conjugation of a cytotoxic agent to the cell-binding agents having linkers bound thereto, and/or after a purification step. When the holding step comprises holding the mixture after conjugation of a cytotoxic agent to the cell-binding agents having linkers bound thereto and/or after a purification step following the conjugation step, the mixture can be subjected to an ion exchange chromatography membrane before or after the holding step, or both. In one embodiment, the process comprises subjecting a mixture comprising a cell-binding agent cytotoxic agent conjugate and one or more impurities to a holding step after the conjugation step, wherein the mixture is subjected to an ion exchange chromatography membrane after the holding step and prior to the purification step. In another embodiment, the process comprises subjecting a mixture comprising a cell-binding agent cytotoxic agent conjugate and one or more impurities to a holding step after the conjugation step, wherein the mixture is subjected to a purification step after the holding step, followed by subjecting the mixture to an ion exchange chromatography membrane. The mixture optionally may be subjected to a second holding step prior to subjecting the mixture to the ion exchange chromatography membrane.

The holding step comprises maintaining the solution at a suitable temperature (e.g., about 2° C. to about 37° C.) for a suitable period of time (e.g., about 1 hour to about 1 week) to release the unstably bound linkers from the cell-binding agent while not substantially releasing the stably bound linkers from the cell-binding agent. In one embodiment, the holding step comprises maintaining the solution at a low temperature (e.g., about 2° C. to about 10° C. or about 4° C.), at room temperature (e.g., about 20° C. to about 30° C. or about 20° C. to about 25° C.), or at an elevated temperature (e.g., about 30° C. to about 37° C.).

The duration of the holding step depends on the temperature at which the holding step is performed. For example, the duration of the holding step can be substantially reduced by performing the holding step at elevated temperature, with the maximum temperature limited by the stability of the cell-binding agent-cytotoxic agent conjugate. The holding step can comprise maintaining the solution for about 1 hour to about 1 day (e.g., about 1 hour, about 2 hours, about 3 hours, about 4 hours, about 5 hours, about 6 hours, about 7 hours, about 8 hours, about 9 hours, about 10 hours, about 12 hours, about 14 hours, about 16 hours, about 18 hours, about 20 hours, about 22 hours, or about 24 hours), about 5 hours to about 1 week, about 12 hours to about 1 week (e.g., about 12 hours, about 16 hours, about 20 hours, about 24 hours, about 2 days, about 3 days, about 4 days, about 5 days, about 6 days, or about 7 days), for about 12 hours to about 1 week (e.g., about 12 hours, about 16 hours, about 20 hours, about 24 hours, about 2 days, about 3 days, about 4 days, about 5 days, about 6 days, or about 7 days), or about 1 day to about 1 week.

In one embodiment, the holding step comprises maintaining the solution at a temperature of about 2° C. to about 8° C. for a period of at least about 12 hours for up to 1 day.

The pH value for the holding step preferably is about 4 to about 9 (e.g., about 4.5 to about 8.5 or about 5 to about 8). In one embodiment, the pH values for the holding step range from about 5 to about 7.5 (e.g., about 5.5 to about 7.5, about 6 to about 7.5, about 6.5 to about 7.5, about 7 to about 7.5, about 5 to about 7, about 5 to about 6.5, about 5 to about 5.5, about 5.5 to about 7, about 6 to about 6.5, or about 6 to about 7). For example, pH values for the holding step can be about 4, about 4.5, about 5, about 5.5, about 6, about 6.5, about 7, about 7.5, about 8, about 8.5, or about 9.

The holding step can be performed before or after the cell-binding agent is conjugated to the cytotoxic agent. In one embodiment, the holding step is performed directly after the modification of the cell-binding agent with the bifunctional crosslinking reagent. For example, the inventive process comprises a holding step after modification of the cell-binding agent with a bifunctional crosslinking reagent and before conjugation. After modification of the cell-binding agent, a purification step may be performed before the hold step and/or after the hold step, but prior to the conjugation step. In another embodiment, the holding step is performed directly after conjugation of the cytotoxic agent to the cell-binding agent having linkers bound thereto and prior to purification step. In another embodiment, the holding step is performed after the conjugation and purification steps and followed by an additional purification step.

In specific embodiments, the holding step can comprise incubating the mixture at a pH of about 5-7.5 or about 6.5-7.5 for about 1 hour to about 1 week at about 2° C. to about room temperature.

In one embodiment, the invention provides a process for preparing a cell-binding agent-cytotoxic agent conjugate, which process comprises the addition of exogenous NHS. “Exogenous NHS,” as used herein, refers to NHS that is added during the process from an external source, and does not refer to NHS that is generated during the modification reaction as a result of hydrolysisaminolysis of the bifunctional linker.

In one embodiment, the invention provides a process for preparing a cell-binding agent-cytotoxic agent conjugate, which process comprises the addition of about 0.1 mM to about 300 mM exogenous NHS. For example, the inventive process comprises the addition of about 0.1 mM, about 0.2 mM, about 0.3 mM, about 0.4 mM, about 0.5 mM, about 0.6 mM, about 0.7 mM, about 0.8 mM, about 0.9 mM, about 1.0 mM, about 1.1 mM, about 1.3 mM, about 1.5 mM, about 1.7 mM, about 1.9 mM, about 2.0 mM, about 2.1 mM, about 2.3 mM, about 2.5 mM, about 2.7 mM, about 2.9 mM, about 3.0 mM, about 3.1 mM, about 3.3 mM, about 3.5 mM, about 3.7 mM, about 3.9 mM, about 4.0 mM, about 4.1 mM, about 4.3 mM, about 4.5 mM, about 4.7 mM, about 4.9 mM, about 5.0 mM, about 5.1 mM, about 5.3 mM, about 5.5 mM, about 5.7 mM, about 5.9 mM, about 6.0 mM, about 6.1 mM, about 6.3 mM, about 6.5 mM, about 6.7 mM, about 6.9 mM, about 7.0 mM, about 7.1 mM, about 7.3 mM, about 7.5 mM, about 7.7 mM, about 7.9 mM, about 8.0 mM, about 8.1 mM, about 8.3 mM, about 8.5 mM, about 8.7 mM, about 8.9 mM, about 9.0 mM, about 9.1 mM, about 9.3 mM, about 9.5 mM, about 9.7 mM, about 9.9 mM, about 10 mM, about 11 mM, about 12 mM, about 13 mM, about 14 mM, about 15 mM, about 16 mM, about 17 mM, about 18 mM, about 19 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, about 100 mM, about 110 mM, about 120 mM, about 130 mM, about 140 mM, about 150 mM, about 160 mM, about 170 mM, about 180 mM, about 190 mM, about 200 mM, about 210 mM, about 220 mM, about 230 mM, about 240 mM, about 250 mM, about 260 mM, about 270 mM, about 280 mM, about 290 mM, or about 300 mM exogenous NHS. In one embodiment, the inventive process comprises the addition of about 0.1 mM to about 5 mM, about 0.1 mM to about 10 mM, about 1.0 mM to about 5 mM, about 1.0 mM to about 10 mM, about 5.0 mM to about 10 mM, about 10 mM to about 20 mM, about 20 mM to about 30 mM, about 30 mM to about 40 mM, about 40 mM to about 50 mM, about 50 mM to about 60 mM, about 60 mM to about 70 mM, about 70 mM to about 80 mM, about 80 mM to about 90 mM, about 90 mM to about 100 mM, about 100 mM to about 110 mM, about 110 mM to about 120 mM, about 120 mM to about 130 mM, about 130 mM to about 140 mM, about 140 mM to about 150 mM, about 150 mM to about 160 mM, about 160 mM to about 170 mM, about 170 mM to about 180 mM, about 180 mM to about 190 mM, about 190 mM to about 200 mM, about 200 mM to about 220 mM, about 220 mM to about 240 mM, about 240 mM to about 260 mM, about 260 mM to about 280 mM, or about 280 mM to about 300 mM exogenous NHS. In another embodiment, the inventive process comprises the addition of about 10 mM to about 200 mM, about 20 to about 150 mM, about 50 to about 150 mM, or about 20 to about 100 mM exogenous NHS.

In some embodiments, the inventive process comprises the addition of a molar ratio of exogenous NHS with respect to the amount of NHS generated during the modification reaction as a result of hydrolysisaminolysis of the bifunctional linker. One of ordinary skill in the art can determine the amount of NHS generated during a particular modification as the amount of NHS generated is essentially the same as the amount of the bifunctional linker used. The skilled person can then add a molar ratio of exogenous NHS to the reaction mixture with respect to the amount of NHS generated during the modification reaction. In one embodiment, about 2 to about 200 fold exogenous NHS is added with respect to the amount of NHS generated during the modification reaction. For example, the inventive process comprises adding about 2, about 5, about 10, about 15, about 20, about 25, about 50, about 100, or about 200 fold exogenous NHS with respect to the amount of NHS generated during the modification reaction.

In some embodiments, the inventive process comprises the addition of a molar ratio of exogenous NHS with respect to the amount of the bifunctional linker. In one embodiment, the molar ratio of the exogenous NHS to the bifunctional crosslinking agent is about 0.5 to about 1000 (e.g., about 1 to about 900, about 5 to about 750, about 50 to about 500, about 100 to about 500, about 0.5 to about 500, or about 100 to about 1000. For example, the inventive process comprises about 0.5, about 1, about 2, about 5, about 10, about 15, about 20, about 25, about 50, about 100, about 200, about 300, about 400, about 500, about 600, about 700, about 800, about 900, or about 1000 fold NHS with respect to the amount of the bifunctional linker.

The inventive process comprises the addition of exogenous NHS at any point during a process preparing a cell-binding agent-cytotoxic agent conjugate. For example, the inventive process comprises the addition of exogenous NHS to the modification step (i.e., the step in which a cell-binding agent is reacted with a bifunctional linker), to the conjugation step (i.e., the step in which a modified cell-binding agent is reacted with a cytotoxic agent), to a purification step, or to a holding step between any of the foregoing steps. In one embodiment, the inventive process comprises the addition of exogenous NHS to the modification step (i.e., NHS is added to the modification reaction), to a holding step between the modification step and a purification step, to a holding step between the modification step and the conjugation step, to a purification step, to the conjugation step, to a holding step between the conjugation step and a purification step, and/or to a holding step between two purification steps.

In one embodiment, the invention provides a process for preparing a cell-binding agent having a linker bound thereto, which process comprises contacting a cell-binding agent with a bifunctional crosslinking reagent in the presence of exogenous NHS to covalently attach a linker to the cell-binding agent and thereby prepare a mixture comprising cell-binding agents having linkers bound thereto.

The invention provides a process for preparing compositions of stable conjugates comprising a cell-binding agent chemically coupled to a cytotoxic agent, wherein the compositions are substantially free of unstable conjugates. In this respect, the invention provides a process for preparing cell-binding agent-cytotoxic agent conjugate of substantially high purity and stability. Such compositions can be used for treating diseases because of the high purity and stability of the conjugates. Compositions comprising a cell-binding agent, such as an antibody, chemically coupled to a cytotoxic agent, such as a maytansinoid, are described in, for example, U.S. Pat. No. 7,374,762. In one aspect of the invention, a cell-binding agent-cytotoxic agent conjugate of substantially high purity has one or more of the following features: (a) greater than about 90% (e.g., greater than or equal to about 91%, 92%, 93%, 94%, 95%, 96%, 97%, 98%, 99%, or 100%), preferably greater than about 95%, of conjugate species are monomeric, (b) unconjugated linker level in the conjugate preparation or purified conjugate is less than about 10% (e.g., less than or equal to about 9%, 8%, 7%, 6%, 5%, 4%, 3%, 2%, 1%, or 0%) (relative to total linker), (c) less than 10% of conjugate species are crosslinked (e.g., less than or equal to about 9%, 8%, 7%, 6%, 5%, 4%, 3%, 2%, 1%, or 0%), (d) free cytotoxic agent level in the conjugate preparation or purified conjugate is less than about 5%, less than about 3%, less than about 2% (e.g., less than or equal to about 1.9%, 1.8%, 1.7%, 1.6%, 1.5%, 1.4%, 1.3%, 1.2%, 1.1%, 1.0%, 0.9%, 0.8%, 0.7%, 0.6%, 0.5%, 0.4%, 0.3%, 0.2%, 0.1%, or 0%) (relative to total cytotoxic agent), (f) cytotoxic dimer species level in the conjugate preparation or purified conjugate is less than about 5%, less than about 3%, less than about 2% (e.g., less than or equal to about 1.9%, 1.8%, 1.7%, 1.6%, 1.5%, 1.4%, 1.3%, 1.2%, 1.1%, 1.0%, 0.9%, 0.8%, 0.7%, 0.6%, 0.5%, 0.4%, 0.3%, 0.2%, 0.1%, or 0%) (relative to total cytotoxic agent), and/or (e) no substantial increase in free cytotoxic agent level upon storage (e.g., after about 1 week, about 2 weeks, about 3 weeks, about 1 month, about 2 months, about 3 months, about 4 months, about 5 months, about 6 months, about 1 year, about 2 years, or about 5 years). “Substantial increase” in free cytotoxic agent level means that after certain storage time, the increase in the level of free cytotoxic agent is less than about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, about 2.0%, about 2.2%, about 2.5%, about 2.7%, about 3.0%, about 3.2%, about 3.5%, about 3.7%, or about 4.0%.

As used herein, the term “unconjugated linker” refers to the cell-binding agent that is covalently linked with the bifunctional crosslinking reagent, wherein the cell-binding agent is not covalently coupled to the cytotoxic agent through the linker of the bifunctional crosslinking reagent (i.e., the “unconjugated linker” can be represented by CBA-L, wherein CBA represents the cell-binding agent and L represents the bifunctional crosslinking reagent. In contrast, the cell-binding agent cytotoxic agent conjugate can be represented by CBA-L-D, wherein D represents the cytotoxic agent).

As used herein, the term “cytotoxic agent dimers” refers to dimers comprising free cytotoxic agent, wherein the cytotoxic agent is not chemically coupled to the cell-binding agent through the linker. In one embodiment, the cytotoxic agent dimers are chemically coupled to each other through a linker (i.e., the “cytotoxic agent dimers” can be represented by D-L-D, wherein D represents the cytotoxic agent and L represents the bifunctional crosslinking reagent. In contrast, the cell-binding agent cytotoxic agent conjugate can be represented by CBA-L-D, wherein CBA represents the cell-binding agent). In another embodiment, the cytotoxic agent dimers are not chemically coupled to each other through a linker (i.e., the “cytotoxic agent dimers” can be represented by D-D, wherein D represents the cytotoxic agent. In contrast, the cell-binding agent cytotoxic agent conjugate can be represented by CBA-L-D, wherein CBA represents the cell-binding agent and L represents the bifunctional crosslinking reagent). In one embodiment, some of the cytotoxic agent dimers are chemically coupled to each other through a linker and some of the cytotoxic agent dimers are not chemically coupled to each other through a linker (i.e., the “cytotoxic agent dimers” can be represented by D-L-D and D-D, wherein D represents the cytotoxic agent and L represents the bifunctional crosslinking reagent).

In one embodiment, the cytotoxic agent dimers are DM1-DM1 dimers and DM1-MCC-DM1 dimers, when the linker is SMCC and the cytotoxic agent is DM1.

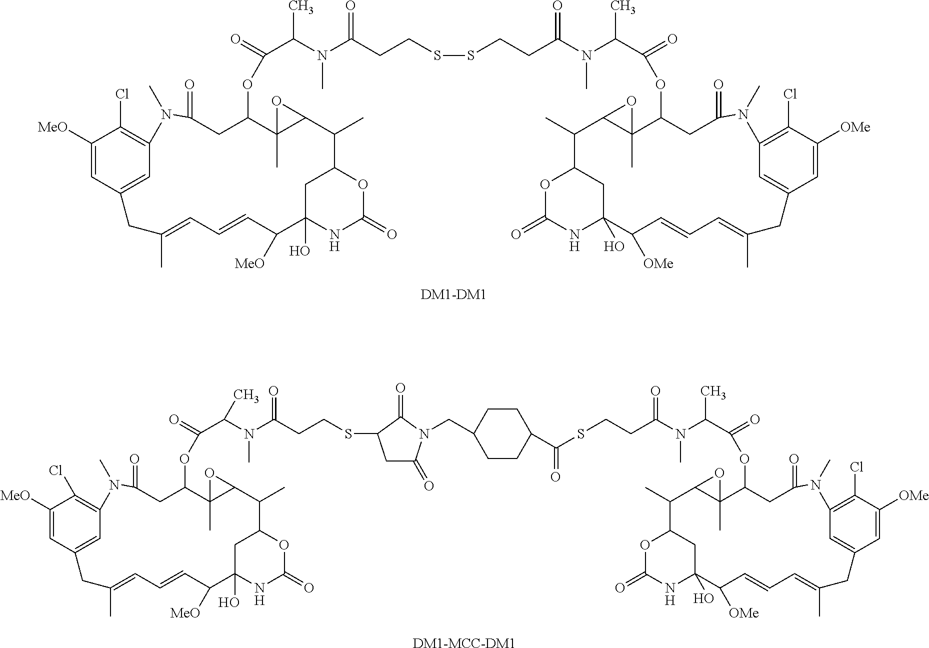

In another embodiment, the cytotoxic agent dimers are DM1-DM1 dimers and DM1-SPP-DM1 dimers, when the linker is SPP and the cytotoxic agent is DM1.

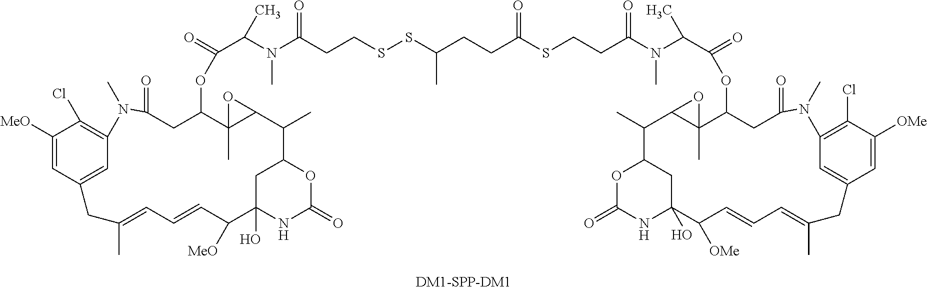 [SMILES: COc1cc2cc(c1Cl)N(C)C(=O)CC(OC(=O)C(C)N(C)C(=O)CCSSC(C)CCC(=O)SCCC(=O)N(C)C(C)C(=O)OC1CC(=O)N(C)c3cc(cc(OC)c3Cl)C/C(C)=C/C=C/C(OC)C3(O)CC(OC(=O)N3)C(C)C3OC13C)C1(C)OC1C(C)C1CC(O)(NC(=O)O1)C(OC)/C=C/C=C(\C)C2]

In another embodiment, the cytotoxic agent dimers are DM1-DM1 dimers and DM1-CX1-1-DM1 dimers, when the linker is CX1-1 and the cytotoxic agent is DM1.

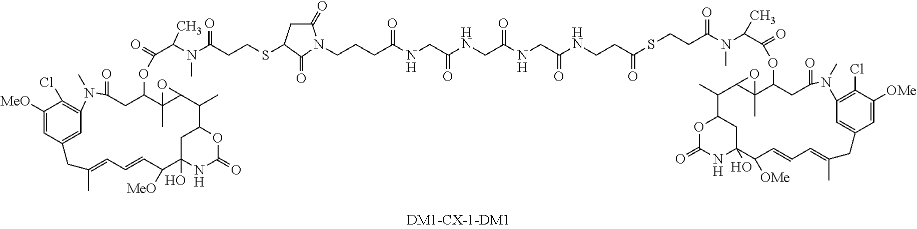 [SMILES: COc1cc2cc(c1Cl)N(C)C(=O)CC(OC(=O)C(C)N(C)C(=O)CCSC(=O)CCNC(=O)CNC(=O)CNC(=O)CNC(=O)CCCN1C(=O)CC(SCCC(=O)N(C)C(C)C(=O)OC3CC(=O)N(C)c4cc(cc(OC)c4Cl)C/C(C)=C/C=C/C(OC)C4(O)CC(OC(=O)N4)C(C)C4OC34C)C1=O)C1(C)OC1C(C)C1CC(O)(NC(=O)O1)C(OC)/C=C/C=C(\C)C2]

As used herein, the term “free cytotoxic agent” refers to any form of the cytotoxic agent that is not chemically coupled to the cell-binding agent through the linker (i.e., the “free cytotoxic agent” can include, but is not limited to, the cytotoxic agent alone represented by D, the cytotoxic agent coupled with the linker or linker derivatives (e.g., hydrolyzed derivatives) represented by D-L, and cytotoxic agent dimers represented by D-D and D-L-D described above).

In one embodiment, the free cytotoxic agent includes DM1, MCC-DM1, hydro-SMCC-DM1, SMCC-DM1, DM1-SPP, DM1-TPA, DM1-DM1, DM1-MCC-DM1, and DM1-SPP-DM1.

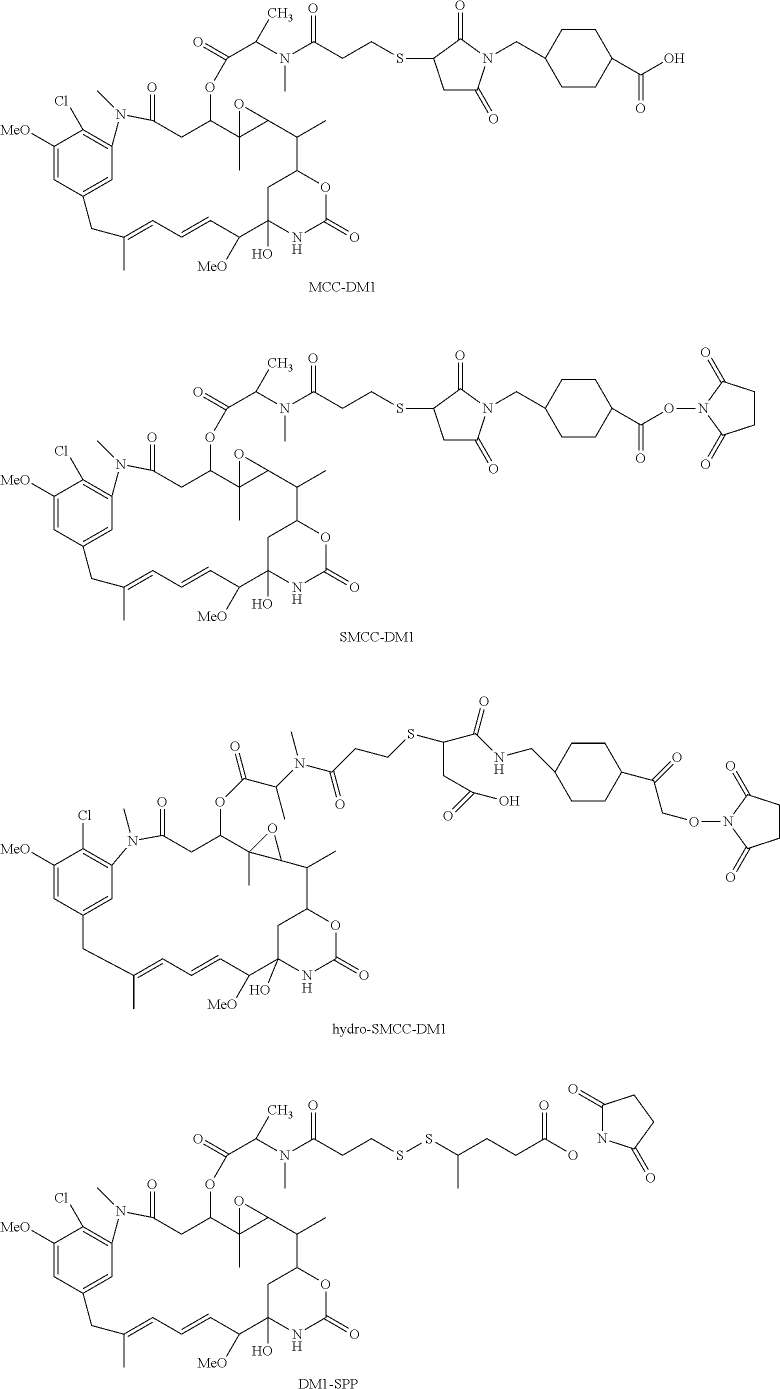

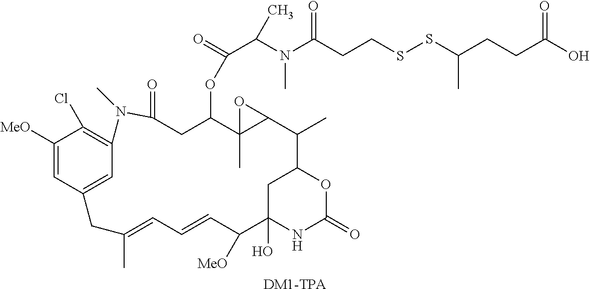

In another embodiment, the free cytotoxic agent includes DM4, DM4-sulfo-SPDB, hydrolyzed DM4-sulfo-SPDB, DM4-SPY, and DM4-sulfo-TBA.

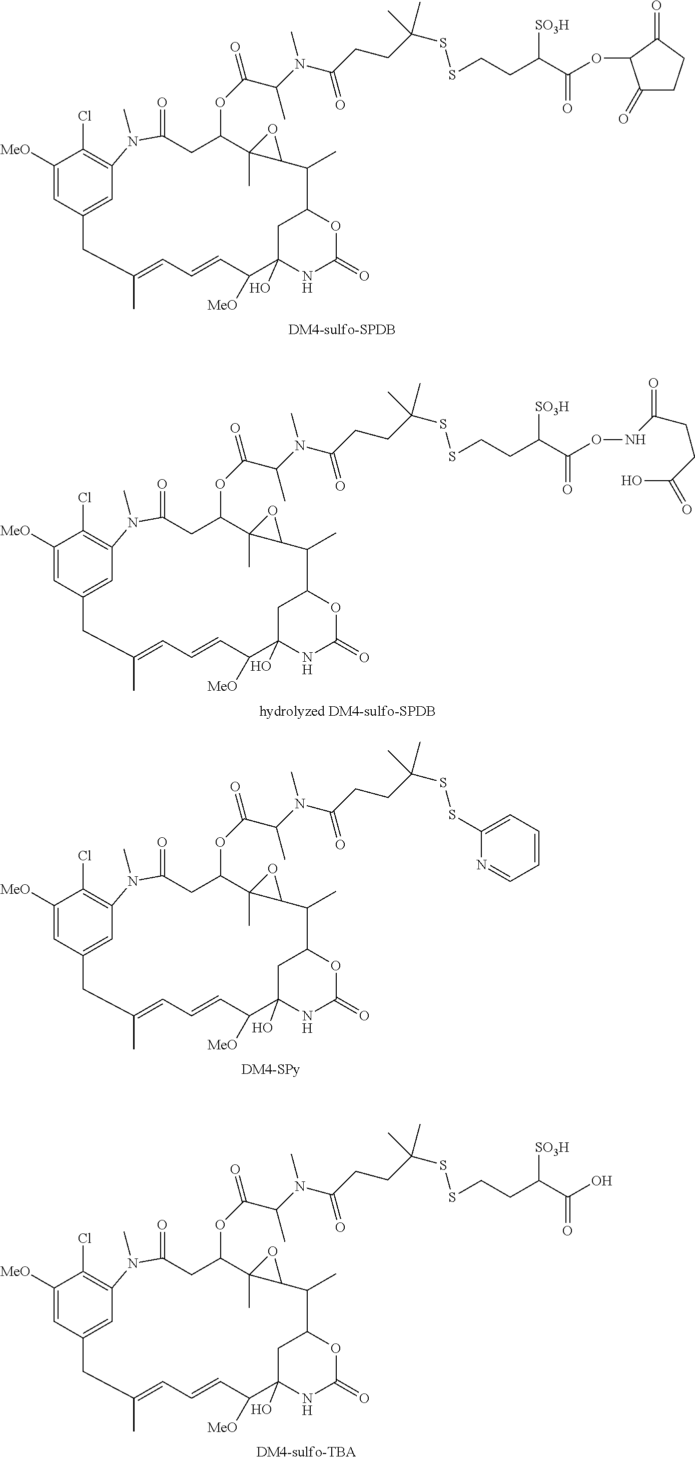

As used herein, the term “aggregates of the cell-binding agent cytotoxic agent conjugate” refers to two or more cell-binding agent cytotoxic agent conjugates covalently or noncovalently coupled to each other (e.g., two or more cell-binding agent cytotoxic agent conjugates covalently coupled through the linker).

In one embodiment, the average molar ratio of the cytotoxic agent to the cell-binding agent in the cell-binding agent cytotoxic agent conjugate is about 1 to about 10, about 2 to about 7, about 3 to about 5, about 2.5 to about 4.5 (e.g., about 2.5, about 2.6, about 2.7, about 2.8, about 2.9, about 3.0, about 3.1, about 3.3, about 3.4, about 3.5, about 3.6, about 3.7, about 3.8, about 3.9, about 4.0, about 4.1, about 4.2, about 4.3, about 4.4, about 4.5), about 3.0 to about 4.0, about 3.2 to about 4.2, about 4.5 to 5.5 (e.g., about 4.5, about 4.6, about 4.7, about 4.8, about 4.9, about 5.0, about 5.1, about 5.2, about 5.3, about 5.4, about 5.5).

The cell-binding agent can be any suitable agent that binds to a cell, typically and preferably an animal cell (e.g., a human cell). The cell-binding agent preferably is a peptide or a polypeptide or a glycotope. Suitable cell-binding agents include, for example, antibodies (e.g., monoclonal antibodies and fragments thereof), interferons (e.g., alpha, beta, gamma), lymphokines (e.g., interleukin 2 (IL-2), interleukin 3 (IL-3), interleukin 4 (IL-4), interleukin 6 (IL-6), hormones (e.g., insulin, TRH (thyrotropin releasing hormone), MSH (melanocyte-stimulating hormone), steroid hormones, such as androgens and estrogens), growth factors and colony-stimulating factors, such as epidermal growth factor (EGF), transforming growth factor alpha (TGF-alpha), fibroblast growth factor (FGF), vascular endothelial growth factor (VEGF), colony stimulating factors (CSFs), such as G-CSF, M-CSF and GM-CSF (Burgess, Immunology Today 5:155-158 (1984)), nutrient-transport molecules (e.g., transferrin), vitamins (e.g., folate) and any other agent or molecule that specifically binds a target molecule on the surface of a cell.

Where the cell-binding agent is an antibody, it binds to an antigen that is a polypeptide and may be a transmembrane molecule (e.g., receptor) or a ligand, such as a growth factor. Exemplary antigens include molecules such as renin; a growth hormone, including human growth hormone and bovine growth hormone; growth hormone releasing factor; parathyroid hormone; thyroid stimulating hormone; lipoproteins; alpha-1-antitrypsin; insulin A-chain; insulin B-chain; proinsulin; follicle stimulating hormone; calcitonin; luteinizing hormone; glucagon; clotting factors such as factor vmc, factor IX, tissue factor (TF), and von Willebrands factor; anti-clotting factors such as Protein C; atrial natriuretic factor; lung surfactant; a plasminogen activator, such as urokinase or human urine or tissue-type plasminogen activator (t-PA); bombesin; thrombin; hemopoietic growth factor; tumor necrosis factor-alpha and -beta; enkephalinase; RANTES (regulated on activation normally T-cell expressed and secreted); human macrophage inflammatory protein (MIP-1-alpha); a serum albumin, such as human serum albumin; Muellerian-inhibiting substance; relaxin A-chain; relaxin B-chain; prorelaxin; mouse gonadotropin-associated peptide; a microbial protein, such as beta-lactamase; DNase; IgE; a cytotoxic T-lymphocyte associated antigen (CTLA), such as CTLA-4; inhibin; activin; vascular endothelial growth factor (VEGF); receptors for hormones or growth factors; protein A or D; rheumatoid factors; a neurotrophic factor such as bone-derived neurotrophic factor (BDNF), neurotrophin-3, -4, -5, or -6 (NT-3, NT4, NT-5, or NT-6), or a nerve growth factor such as NGF-β; platelet-derived growth factor (PDGF); fibroblast growth factor (FGF) such as aFGF and bFGF; fibroblast growth factor receptor such as FGFR24 and FGFR3, epidermal growth factor (EGF); transforming growth factor (TGF) such as TGF-alpha and TGF-beta, including TGF-β1, TGF-β2, TGF-P3, TGF-β4, or TGF-β5; insulin-like growth factor-I and -II (IGF-I and IGF-II); des(1-3)-IGF-I (brain IGF-I), insulin-like growth factor binding proteins, EpCAM, GD3, FLT3, PSMA, PSCA, MUC1, MUC16, STEAP, CEA, TENB2, EphA receptors, EphB receptors, folate receptor, FOLRl, mesothelin, cripto, alphaᵥbeta₆, integrins, VEGF, VEGFR, EGFR, transferrin receptor, IRTA1, IRTA2, IRTA3, IRTA4, IRTA5; CD proteins such as CD2, CD3, CD4, CD5, CD6, CD8, CD11, CD14, CD19, CD20, CD21, CD22, CD25, CD26, CD28, CD30, CD33, CD36, CD37, CD38, CD40, CD44, CD52, CD55, CD56, CD59, CD70, CD79, CD80. CD81, CD103, CD105, CD134, CD137, CD138, CD152 or an antibody which binds to one or more tumor-associated antigens or cell-surface receptors disclosed in U.S. Patent Application Publication No. 20080171040 or U.S. Patent Application Publication No. 20080305044 and are incorporated in their entirety by reference; erythropoietin; osteoinductive factors; immunotoxins; a bone morphogenetic protein (BMP); an interferon, such as interferon-alpha, -beta, and -gamma; colony stimulating factors (CSFs), e.g., M-CSF, GM-CSF, and G-CSF; interleukins (ILs), e.g., IL-1 to IL-10; superoxide dismutase; T-cell receptors; surface membrane proteins; decay accelerating factor; viral antigen such as, for example, a portion of the HIV envelope; transport proteins; homing receptors; addressins; regulatory proteins; integrins, such as CD11a, CD11b, CD11c, CD18, an ICAM, VLA-4 and VCAM; a tumor associated antigen such as HER2, HER3 or HER4 receptor; endoglin, c-Met, IGF1R, prostate antigens such as PCA3, PSA, PSGR, NGEP, PSMA, PSCA, TMEFF2, and STEAP1; LGR5, B7H4, and fragments of any of the above-listed polypeptides.

Additionally, GM-CSF, which binds to myeloid cells can be used as a cell-binding agent to diseased cells from acute myelogenous leukemia. IL-2 which binds to activated T-cells can be used for prevention of transplant graft rejection, for therapy and prevention of graft-versus-host disease, and for treatment of acute T-cell leukemia. MSH, which binds to melanocytes, can be used for the treatment of melanoma, as can antibodies directed towards melanomas. Folic acid can be used to target the folate receptor expressed on ovarian and other tumors. Epidermal growth factor can be used to target squamous cancers such as lung and head and neck. Somatostatin can be used to target neuroblastomas and other tumor types.

Cancers of the breast and testes can be successfully targeted with estrogen (or estrogen analogues) or androgen (or androgen analogues) respectively as cell-binding agents

The term “antibody,” as used herein, refers to any immunoglobulin, any immunoglobulin fragment, such as Fab, Fab′, F(ab′)₂, dsFv, sFv, minibodies, diabodies, tribodies, tetrabodies (Parham, J. Immunol. 131: 2895-2902 (1983); Spring et al. J. Immunol. 113: 470-478 (1974); Nisonoff et al. Arch. Biochem. Biophys. 89: 230-244 (1960), Kim et al., Mol. Cancer Ther., 7: 2486-2497 (2008), Carter, Nature Revs., 6: 343-357 (2006)), or immunoglobulin chimera, which can bind to an antigen on the surface of a cell (e.g., which contains a complementarity determining region (CDR)). Any suitable antibody can be used as the cell-binding agent. One of ordinary skill in the art will appreciate that the selection of an appropriate antibody will depend upon the cell population to be targeted. In this regard, the type and number of cell surface molecules (i.e., antigens) that are selectively expressed in a particular cell population (typically and preferably a diseased cell population) will govern the selection of an appropriate antibody for use in the inventive composition. Cell surface expression profiles are known for a wide variety of cell types, including tumor cell types, or, if unknown, can be determined using routine molecular biology and histochemistry techniques.

The antibody can be polyclonal or monoclonal, but is most preferably a monoclonal antibody. As used herein, “polyclonal” antibodies refer to heterogeneous populations of antibody molecules, typically contained in the sera of immunized animals. “Monoclonal” antibodies refer to homogenous populations of antibody molecules that are specific to a particular antigen. Monoclonal antibodies are typically produced by a single clone of B lymphocytes (“B cells”). Monoclonal antibodies may be obtained using a variety of techniques known to those skilled in the art, including standard hybridoma technology (see, e.g., Köhler and Milstein, Eur. J. Immunol., 5: 511-519 (1976), Harlow and Lane (eds.), Antibodies: A Laboratory Manual, CSH Press (1988), and C. A. Janeway et al. (eds.), Immunobiology, 5^{th} Ed., Garland Publishing, New York, N.Y. (2001)). In brief, the hybridoma method of producing monoclonal antibodies typically involves injecting any suitable animal, typically and preferably a mouse, with an antigen (i.e., an “immunogen”). The animal is subsequently sacrificed, and B cells isolated from its spleen are fused with human myeloma cells. A hybrid cell is produced (i.e., a “hybridoma”), which proliferates indefinitely and continuously secretes high titers of an antibody with the desired specificity in vitro. Any appropriate method known in the art can be used to identify hybridoma cells that produce an antibody with the desired specificity. Such methods include, for example, enzyme-linked immunosorbent assay (ELISA), Western blot analysis, and radioimmunoassay. The population of hybridomas is screened to isolate individual clones, each of which secretes a single antibody species to the antigen. Because each hybridoma is a clone derived from fusion with a single B cell, all the antibody molecules it produces are identical in structure, including their antigen binding site and isotype. Monoclonal antibodies also may be generated using other suitable techniques including EBV-hybridoma technology (see, e.g., Haskard and Archer, J. Immunol. Methods, 74(2): 361-67 (1984), and Roder et al., Methods Enzymol., 121: 140-67 (1986)), bacteriophage vector expression systems (see, e.g., Huse et al., Science, 246: 1275-81 (1989)), or phage display libraries comprising antibody fragments, such as Fab and scFv (single chain variable region) (see, e.g., U.S. Pat. Nos. 5,885,793 and 5,969,108, and International Patent Application Publications WO 9201047 and WO 9906587).

The monoclonal antibody can be isolated from or produced in any suitable animal, but is preferably produced in a mammal, more preferably a mouse or human, and most preferably a human. Methods for producing an antibody in mice are well known to those skilled in the art and are described herein. With respect to human antibodies, one of ordinary skill in the art will appreciate that polyclonal antibodies can be isolated from the sera of human subjects vaccinated or immunized with an appropriate antigen. Alternatively, human antibodies can be generated by adapting known techniques for producing human antibodies in non-human animals such as mice (see, e.g., U.S. Pat. Nos. 5,545,806, 5,569,825, and 5,714,352, and U.S. Patent Application Publication No. 20020197266 A1).

While being the ideal choice for therapeutic applications in humans, human antibodies, particularly human monoclonal antibodies, typically are more difficult to generate than mouse monoclonal antibodies. Mouse monoclonal antibodies, however, induce a rapid host antibody response when administered to humans, which can reduce the therapeutic or diagnostic potential of the antibody-cytotoxic agent conjugate. To circumvent these complications, a monoclonal antibody preferably is not recognized as “foreign” by the human immune system.

To this end, phage display can be used to generate the antibody. In this regard, phage libraries encoding antigen-binding variable (V) domains of antibodies can be generated using standard molecular biology and recombinant DNA techniques (see, e.g., Sambrook et al. (eds.), Molecular Cloning, A Laboratory Manual, 3^{rd} Edition, Cold Spring Harbor Laboratory Press, New York (2001)). Phage encoding a variable region with the desired specificity are selected for specific binding to the desired antigen, and a complete human antibody is reconstituted comprising the selected variable domain. Nucleic acid sequences encoding the reconstituted antibody are introduced into a suitable cell line, such as a myeloma cell used for hybridoma production, such that human antibodies having the characteristics of monoclonal antibodies are secreted by the cell (see, e.g., Janeway et al., supra, Huse et al., supra, and U.S. Pat. No. 6,265,150). Alternatively, monoclonal antibodies can be generated from mice that are transgenic for specific human heavy and light chain immunoglobulin genes. Such methods are known in the art and described in, for example, U.S. Pat. Nos. 5,545,806 and 5,569,825, and Janeway et al., supra.

Most preferably the antibody is a humanized antibody. As used herein, a “humanized” antibody is one in which the complementarity-determining regions (CDR) of a mouse monoclonal antibody, which form the antigen binding loops of the antibody, are grafted onto the framework of a human antibody molecule. Owing to the similarity of the frameworks of mouse and human antibodies, it is generally accepted in the art that this approach produces a monoclonal antibody that is antigenically identical to a human antibody but binds the same antigen as the mouse monoclonal antibody from which the CDR sequences were derived. Methods for generating humanized antibodies are well known in the art and are described in detail in, for example, Janeway et al., supra, U.S. Pat. Nos. 5,225,539, 5,585,089 and 5,693,761, European Patent No. 0239400 B1, and United Kingdom Patent No. 2188638. Humanized antibodies can also be generated using the antibody resurfacing technology described in U.S. Pat. No. 5,639,641 and Pedersen et al., J. Mol. Biol., 235: 959-973 (1994). While the antibody employed in the conjugate of the inventive composition most preferably is a humanized monoclonal antibody, a human monoclonal antibody and a mouse monoclonal antibody, as described above, are also within the scope of the invention.

Antibody fragments that have at least one antigen binding site, and thus recognize and bind to at least one antigen or receptor present on the surface of a target cell, also are within the scope of the invention. In this respect, proteolytic cleavage of an intact antibody molecule can produce a variety of antibody fragments that retain the ability to recognize and bind antigens. For example, limited digestion of an antibody molecule with the protease papain typically produces three fragments, two of which are identical and are referred to as the Fab fragments, as they retain the antigen binding activity of the parent antibody molecule. Cleavage of an antibody molecule with the enzyme pepsin normally produces two antibody fragments, one of which retains both antigen-binding arms of the antibody molecule, and is thus referred to as the F(ab′)₂ fragment. Reduction of a F(ab′)₂ fragment with dithiothreitol or mercaptoethylamine produces a fragment referred to as a Fab′ fragment. A single-chain variable region fragment (sFv) antibody fragment, which consists of a truncated Fab fragment comprising the variable (V) domain of an antibody heavy chain linked to a V domain of a light antibody chain via a synthetic peptide, can be generated using routine recombinant DNA technology techniques (see, e.g., Janeway et al., supra). Similarly, disulfide-stabilized variable region fragments (dsFv) can be prepared by recombinant DNA technology (see, e.g., Reiter et al., Protein Engineering, 7: 697-704 (1994)). Antibody fragments in the context of the invention, however, are not limited to these exemplary types of antibody fragments. Any suitable antibody fragment that recognizes and binds to a desired cell surface receptor or antigen can be employed. Antibody fragments are further described in, for example, Parham, J. Immunol., 131: 2895-2902 (1983), Spring et al., J. Immunol., 113: 470-478 (1974), and Nisonoff et al., Arch. Biochem. Biophys., 89: 230-244 (1960). Antibody-antigen binding can be assayed using any suitable method known in the art, such as, for example, radioimmunoassay (RIA), ELISA, Western blot, immunoprecipitation, and competitive inhibition assays (see, e.g., Janeway et al., supra, and U.S. Patent Application Publication No. 20020197266 A1).

In addition, the antibody can be a chimeric antibody or an antigen binding fragment thereof. By “chimeric” it is meant that the antibody comprises at least two immunoglobulins, or fragments thereof, obtained or derived from at least two different species (e.g., two different immunoglobulins, such as a human immunoglobulin constant region combined with a murine immunoglobulin variable region). The antibody also can be a domain antibody (dAb) or an antigen binding fragment thereof, such as, for example, a camelid antibody (see, e.g., Desmyter et al., Nature Struct. Biol., 3: 752, (1996)), or a shark antibody, such as, for example, a new antigen receptor (IgNAR) (see, e.g., Greenberg et al., Nature, 374: 168 (1995), and Stanfield et al., Science, 305: 1770-1773 (2004)).

Any suitable antibody can be used in the context of the invention. For example, the monoclonal antibody J5 is a murine IgG2a antibody that is specific for Common Acute Lymphoblastic Leukemia Antigen (CALLA) (Ritz et al., Nature, 283: 583-585 (1980)), and can be used to target cells that express CALLA (e.g., acute lymphoblastic leukemia cells). The monoclonal antibody MY9 is a murine IgG1 antibody that binds specifically to the CD33 antigen (Griffin et al., Leukemia Res., 8: 521 (1984)), and can be used to target cells that express CD33 (e.g., acute myelogenous leukemia (AML) cells).

Similarly, the monoclonal antibody anti-B4 (also referred to as B4) is a murine IgG1 antibody that binds to the CD19 antigen on B cells (Nadler et al., J. Immunol., 131: 244-250 (1983)), and can be used to target B cells or diseased cells that express CD19 (e.g., non-Hodgkin's lymphoma cells and chronic lymphoblastic leukemia cells). N901 is a murine monoclonal antibody that binds to the CD56 (neural cell adhesion molecule) antigen found on cells of neuroendocrine origin, including small cell lung tumor, which can be used in the conjugate to target drugs to cells of neuroendocrine origin. The J5, MY9, and B4 antibodies preferably are resurfaced or humanized prior to their use as part of the conjugate. Resurfacing or humanization of antibodies is described in, for example, Roguska et al., Proc. Natl. Acad. Sci. USA, 91: 969-73 (1994). In one embodiment, the anti-B4 antibody is huB4. In another embodiment, the anti-B4 antibody comprises a heavy chain and a light chain, wherein the heavy chain has the following sequence

(SEQ ID NO: 1)
QVQLVQPGAE VVKPGASVKL SCKTSGYTFT SNWMHWVKQA
PGQGLEWIGE IDPSDSYTNY NQNFQGKAKL TVDKSTSTAY
MEVSSLRSDD TAVYYCARGS NPYYYAMDYW GQGTSVTVSS
ASTKGPSVFP LAPSSKSTSG GTAALGCLVK DYFPEPVTVS
WNSGALTSGV HTFPAVLQSS GLYSLSSVVT VPSSSLGTQT
YICNVNHKPS NTKVDKKVEP KSCDKTHTCP PCPAPELLGG
PSVFLFPPKP KDTLMISRTP EVTCVVVDVS HEDPEVKFNW
YVDGVEVHNA KTKPREEQYN STYRVVSVLT VLHQDWLNGK
EYKCKVSNKA LPAPIEKTIS KAKGQPREPQ VYTLPPSRDE
LTKNQVSLTC LVKGFYPSDI AVEWESNGQP ENNYKTTPPV
LDSDGSFFLY SKLTVDKSRW QQGNVFSCSV MHEALHNHYT
QKSLSLSPGK

and the light chain has the following sequence

(SEQ ID NO: 2)
EIVLTQSPAI MSASPGERVT MTCSASSGVN YMHWYQQKPG
TSPRRWIYDT SKLASGVPAR FSGSGSGTDY SLTISSMEPE
DAATYYCHQR GSYTFGGGTK LEIKRTVAAP SVFIFPPSDE
QLKSGTASVV CLLNNFYPRE AKVQWKVDNA LQSGNSQESV
TEQDSKDSTY SLSSTLTLSK ADYEKHKVYA CEVTHQGLSS
PVTKSFNRGE C.

In addition, the monoclonal antibody C242 binds to the CanAg antigen (see, e.g., U.S. Pat. No. 5,552,293), and can be used to target the conjugate to CanAg expressing tumors, such as colorectal, pancreatic, non-small cell lung, and gastric cancers. HuC242 is a humanized form of the monoclonal antibody C242 (see, e.g., U.S. Pat. No. 5,552,293). The hybridoma from which HuC242 is produced is deposited with ECACC identification Number 90012601. HuC242 can be prepared using CDR-grafting methodology (see, e.g., U.S. Pat. Nos. 5,585,089, 5,693,761, and 5,693,762) or resurfacing technology (see, e.g., U.S. Pat. No. 5,639,641). HuC242 can be used to target the conjugate to tumor cells expressing the CanAg antigen, such as, for example, colorectal, pancreatic, non-small cell lung, and gastric cancer cells.

To target ovarian cancer and prostate cancer cells, an anti-MUC1 antibody can be used as the cell-binding agent in the conjugate. Anti-MUC1 antibodies include, for example, anti-HMFG-2 (see, e.g., Taylor-Papadimitriou et al., Int. J. Cancer, 28: 17-21 (1981)), hCTM01 (see, e.g., van Hof et al., Cancer Res., 56: 5179-5185 (1996)), and DS6. Prostate cancer cells also can be targeted with the conjugate by using an anti-prostate-specific membrane antigen (PSMA) as the cell-binding agent, such as J591 (see, e.g., Liu et al., Cancer Res., 57: 3629-3634 (1997)). Moreover, cancer cells that express the Her2 antigen, such as breast, prostate, and ovarian cancers, can be targeted with the conjugate by using anti-HER2 antibodies, e.g., trastuzumab, as the cell-binding agent. Cells that express epidermal growth factor receptor (EGFR) and variants thereof, such as the type III deletion mutant, EGFRvIII, can be targeted with the conjugate by using anti-EGFR antibodies. Anti-EGFR antibodies are described in International Patent Application Nos. PCT/U.S. Ser. No. 11/058,385 and PCT/U.S. Ser. No. 11/058,378. Anti-EGFRvIII antibodies are described in U.S. Pat. Nos. 7,736,644 and 7,628,986, and U.S. Patent Application Publications 20100111979; 20090240038; 20090175887; 20090156790; and 20090155282. Anti-IGF-IR antibodies that bind to insulin-like growth factor receptor, such as those described in U.S. Pat. No. 7,982,024, also can be used in the conjugate. Antibodies that bind to CD27L, Cripto, CD138, CD38, EphA2, integrins, CD37, folate, CD20, PSGR, NGEP, PSCA, TMEFF2, STEAP1, endoglin, and Her3 also can be used in the conjugate.

In one embodiment, the antibody is selected from the group consisting of huN901, anti-CD33 antibody (e.g., huMy9-6), huB4, huC242, an anti-HER2 antibody (e.g., trastuzumab), bivatuzumab, sibrotuzumab, rituximab, huDS6, anti-mesothelin antibodies described in International Patent Application Publication WO 2010124797 (such as MF-T), anti-cripto antibodies described in U.S. Patent Application Publication 20100093980 (such as huB3F6), anti-CD138 antibodies described in U.S. Patent Application Publication 20070183971 (such as B-B4 or humanized B-B4 or nBT062), anti-EGFR antibodies described in International Patent Application Publications WO 2012058592 and WO 2012058588 (such as EGFR-7), anti-EGFRvIII antibodies described U.S. Pat. Nos. 7,736,644 and 7,628,986 and U.S. Patent Application Publications 20100111979, 20090240038, 20090175887, 20090156790 and 20090155282, humanized EphA2 antibodies described in International Patent Application Publications WO 2011039721 and WO 2011039724 (such as 2H11R35R74); anti-CD38 antibodies described in International Patent Application Publication WO 2008047242 (such as hu38SB19), anti-folate receptor antibodies described in International Patent Application Publication WO 2011106528, and U.S. Patent Application Publication 20120009181 (e.g., huMov19 version 1.0 or 1.6); anti-IGF1R antibodies described in U.S. Pat. Nos. 5,958,872, 6,596,743, and 7,982,024; anti-CD37 antibodies described in U.S. Patent Application Publication 20110256153 (e.g., huCD37-3 version 1.0); anti-integrin αᵥβ₆ antibodies described in U.S. Patent Application Publication 20060127407 (e.g., CNTO95); and anti-Her3 antibodies described in International Patent Application Publication WO 2012019024. In one embodiment of the invention, the antibody is not huN901, or CNTO95. In one embodiment, the anti-CD37 antibody is huCD37-3, wherein the antibody comprises a variable heavy chain and a variable light chain, wherein the variable heavy chain has the following sequence

(SEQ ID NO: 3)
QVQVQESGPGLVAPSQTLSITCTVSGFSLTTSGVSWVRQPPGKGLEWLG
VIWGDGSTNYHPSLKSRLSIKKDHSKSQVFLKLNSLTAADTATYYCAKG
GYSLAHWGQGTLVTVSS

and the variable light chain has the following sequence

(SEQ ID NO: 4)
DIQMTQSPSSLSVSVGERVTITCRASENIRSNLAWYQQKPGKSPKLLVN
VATNLADGVPSRFSGSGSGTDYSLKINSLQPEDFGTYYCQHYWGTTWTF
GQGTKLEIKR.

While the cell-binding agent preferably is an antibody, the cell-binding agent also can be a non-antibody molecule. Suitable non-antibody molecules include, for example, interferons (e.g., alpha, beta, or gamma interferon), lymphokines (e.g., interleukin 2 (IL-2), IL-3, IL-4, or IL-6), hormones (e.g., insulin), growth factors (e.g., EGF, TGF-alpha, FGF, and VEGF), colony-stimulating factors (e.g., G-CSF, M-CSF, and GM-CSF (see, e.g., Burgess, Immunology Today, 5: 155-158 (1984)), somatostatin, and transferrin (see, e.g., O'Keefe et al., J. Biol. Chem., 260: 932-937 (1985)). For example, GM-CSF, which binds to myeloid cells, can be used as a cell-binding agent to target acute myelogenous leukemia cells. In addition, IL-2, which binds to activated T-cells, can be used for prevention of transplant graft rejection, for therapy and prevention of graft-versus-host disease, and for treatment of acute T-cell leukemia. Epidermal growth factor (EGF) can be used to target squamous cancers such as lung cancer and head and neck cancer. Somatostatin can be used to target neuroblastoma cells and other tumor cell types.

The conjugate can comprise any suitable cytotoxic agent. A “cytotoxic agent,” as used herein, refers to any compound that results in the death of a cell, induces cell death, or decreases cell viability. Suitable cytotoxic agents include, for example, maytansinoids and maytansinoid analogs, taxoids, CC-1065 and CC-1065 analogs, and dolastatin and dolastatin analogs. In a preferred embodiment of the invention, the cytotoxic agent is a maytansinoid, including maytansinol and maytansinol analogs. Maytansinoids are compounds that inhibit microtubule formation and are highly toxic to mammalian cells. Examples of suitable maytansinol analogues include those having a modified aromatic ring and those having modifications at other positions. Such maytansinoids are described in, for example, U.S. Pat. Nos. 4,256,746, 4,294,757, 4,307,016, 4,313,946, 4,315,929, 4,322,348, 4,331,598, 4,361,650, 4,362,663, 4,364,866, 4,424,219, 4,371,533, 4,450,254, 5,475,092, 5,585,499, 5,846,545, and 6,333,410.

Examples of maytansinol analogs having a modified aromatic ring include: (1)C-19-dechloro (U.S. Pat. No. 4,256,746) (prepared by LAH reduction of ansamytocin P2), (2)C-20-hydroxy (or C-20-demethyl) +/−C-19-dechloro (U.S. Pat. Nos. 4,361,650 and 4,307,016) (prepared by demethylation using Streptomyces or Actinomyces or dechlorination using LAH), and (3)C-20-demethoxy, C-20-acyloxy (—OCOR), +/− dechloro (U.S. Pat. No. 4,294,757) (prepared by acylation using acyl chlorides).

Examples of maytansinol analogs having modifications of positions other than an aromatic ring include: (1)C-9-SH (U.S. Pat. No. 4,424,219) (prepared by the reaction of maytansinol with H₂5 or P₂S₅), (2)C-14-alkoxymethyl (demethoxy/CH₂OR) (U.S. Pat. No. 4,331,598), (3)C-14-hydroxymethyl or acyloxymethyl (CH₂OH or CH₂OAc) (U.S. Pat. No. 4,450,254) (prepared from Nocardia), (4)C-15-hydroxyacyloxy (U.S. Pat. No. 4,364,866) (prepared by the conversion of maytansinol by Streptomyces), (5)C-15-methoxy (U.S. Pat. Nos. 4,313,946 and 4,315,929) (isolated from Trewia nudiflora), (6)C-18-N-demethyl (U.S. Pat. Nos. 4,362,663 and 4,322,348) (prepared by the demethylation of maytansinol by Streptomyces), and (7) 4,5-deoxy (U.S. Pat. No. 4,371,533) (prepared by the titanium trichloride/LAH reduction of maytansinol).

In a preferred embodiment of the invention, the conjugate utilizes the thiol-containing maytansinoid DM1, also known as N^{2′}-deacetyl-N^{2′}-(3-mercapto-1-oxopropyl)-maytansine, as the cytotoxic agent. The structure of DM1 is represented by formula (I):

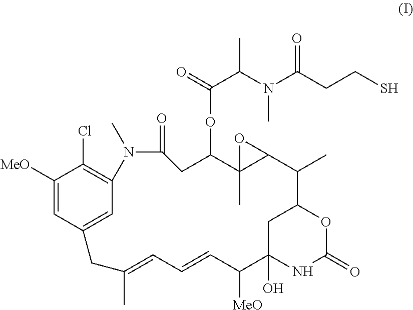 [SMILES: COc1cc2cc(c1Cl)N(C)C(=O)CC(OC(=O)C(C)N(C)C(=O)CCS)C1(C)OC1C(C)C1CC(O)(NC(=O)O1)C(OC)/C=C/C=C(\C)C2]

In another preferred embodiment of the invention, the conjugate utilizes the thiol-containing maytansinoid DM4, also known as N^{2′}-deacetyl-N^{2′}-(4-methyl-4-mercapto-1-oxopentyl)-maytansine, as the cytotoxic agent. The structure of DM4 is represented by formula (II):

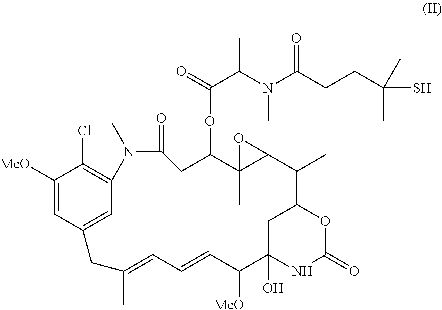 [SMILES: COc1cc2cc(c1Cl)N(C)C(=O)CC(OC(=O)C(C)N(C)C(=O)CCC(C)(C)S)C1(C)OC1C(C)C1CC(O)(NC(=O)O1)C(OC)/C=C/C=C(\C)C2]

Other maytansines may be used in the context of the invention, including, for example, thiol and disulfide-containing maytansinoids bearing a mono or di-alkyl substitution on the carbon atom bearing the sulfur atom. Particularly preferred is a maytansinoid having at the C-3 position (a)C-14 hydroxymethyl, C-15 hydroxy, or C-20 desmethyl functionality, and (b) an acylated amino acid side chain with an acyl group bearing a hindered sulfhydryl group, wherein the carbon atom of the acyl group bearing the thiol functionality has one or two substituents, said substituents being CH₃, C₂H₅, linear or branched alkyl or alkenyl having from 1 to 10 carbon atoms, cyclic alkyl or alkenyl having from 3 to 10 carbon atoms, phenyl, substituted phenyl, or heterocyclic aromatic or heterocycloalkyl radical, and further wherein one of the substituents can be H, and wherein the acyl group has a linear chain length of at least three carbon atoms between the carbonyl functionality and the sulfur atom.

Additional maytansines for use in the context of the invention include compounds represented by formula (III):

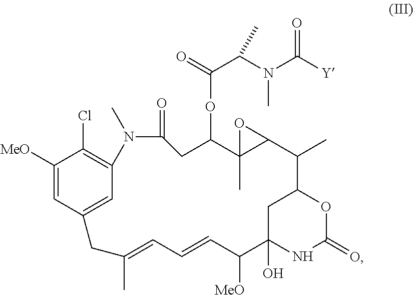

wherein Y′ represents (CR₇R₈)ₗ(CR₉═CR₁₀)ₚ(C≡C)_{q}Aₒ(CR₅R₆)ₘDᵤ(CR₁₁═CR₁₂)ᵣ(C≡C)ₛBₜ(CR₃R₄)ₙCR₁R₂SZ, wherein R₁ and R₂ are each independently CH₃, C₂H₅, linear alkyl or alkenyl having from 1 to 10 carbon atoms, branched or cyclic alkyl or alkenyl having from 3 to 10 carbon atoms, phenyl, substituted phenyl or heterocyclic aromatic or heterocycloalkyl radical, and wherein R₂ also can be H,

wherein A, B, D are cycloalkyl or cycloalkenyl having 3-10 carbon atoms, simple or substituted aryl, or heterocyclic aromatic, or heterocycloalkyl radical,
wherein R₃, R₄, R₅, R₆, R₇, R₈, R₉, R₁₀, R₁₁, and R₁₂ are each independently H, CH₃, C₂H₅, linear alkyl or alkenyl having from 1 to 10 carbon atoms, branched or cyclic alkyl or alkenyl having from 3 to 10 carbon atoms, phenyl, substituted phenyl or heterocyclic aromatic, or heterocycloalkyl radical,
wherein l, m, n, o, p, q, r, s, and t are each independently zero or an integer from 1 to 5, provided that at least two of l, m, n, o, p, q, r, s and t are not zero at any one time, and wherein Z is H, SR or COR, wherein R is linear alkyl or alkenyl having from 1 to 10 carbon atoms, branched or cyclic alkyl or alkenyl having from 3 to 10 carbon atoms, or simple or substituted aryl or heterocyclic aromatic, or heterocycloalkyl radical.

Preferred embodiments of formula (III) include compounds of formula (III) wherein (a) R₁ is H, R₂ is methyl and Z is H, (b) R₁ and R₂ are methyl and Z is H, (c) R₁ is H, R₂ is methyl, and Z is SCH₃, and (d) R₁ and R₂ are methyl, and Z is SCH₃.

Such additional maytansines also include compounds represented by formula (IV-L), (IV-D), or (IV-D,L):

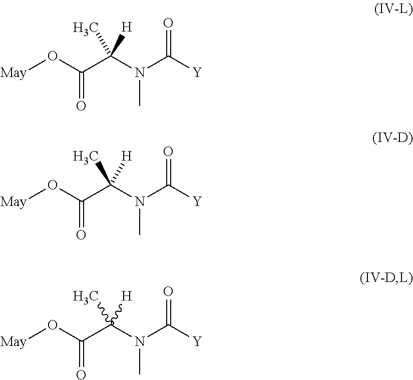

wherein Y represents (CR₇R₈)ₗ(CR₅R₆)ₘ(CR₃R₄)ₙCR₁R₂SZ,
wherein R₁ and R₂ are each independently CH₃, C₂H₅, linear alkyl, or alkenyl having from 1 to 10 carbon atoms, branched or cyclic alkyl or alkenyl having from 3 to 10 carbon atoms, phenyl, substituted phenyl, or heterocyclic aromatic or heterocycloalkyl radical, and wherein R₂ also can be H,
wherein R₃, R₄, R₅, R₆, R₇, and R₈ are each independently H, CH₃, C₂H₅, linear alkyl or alkenyl having from 1 to 10 carbon atoms, branched or cyclic alkyl or alkenyl having from 3 to 10 carbon atoms, phenyl, substituted phenyl, or heterocyclic aromatic or heterocycloalkyl radical,
wherein l, m, and n are each independently an integer of from 1 to 5, and in addition n can be zero,
wherein Z is H, SR, or COR wherein R is linear or branched alkyl or alkenyl having from 1 to 10 carbon atoms, cyclic alkyl or alkenyl having from 3 to 10 carbon atoms, or simple or substituted aryl or heterocyclic aromatic or heterocycloalkyl radical, and
wherein May represents a maytansinoid which bears the side chain at C-3, C-14 hydroxymethyl, C-15 hydroxy, or C-20 desmethyl.

Preferred embodiments of formulas (IV-L), (IV-D) and (IV-D,L) include compounds of formulas (IV-L), (IV-D) and (IV-D,L) wherein (a) R₁ is H, R₂ is methyl, R₅, R₆, R₇, and R₈ are each H, 1 and m are each 1, n is 0, and Z is H, (b) R₁ and R₂ are methyl, R₅, R₆, R₇, R₈ are each H, 1 and m are 1, n is 0, and Z is H, (c) R₁ is H, R₂ is methyl, R₅, R₆, R₇, and R₈ are each H, 1 and m are each 1, n is 0, and Z is SCH₃, or (d) R₁ and R₂ are methyl, R₅, R₆, R₇, R₈ are each H, 1 and m are 1, n is 0, and Z is SCH₃.

Preferably the cytotoxic agent is represented by formula (IV-L).

Additional preferred maytansines also include compounds represented by formula (V):

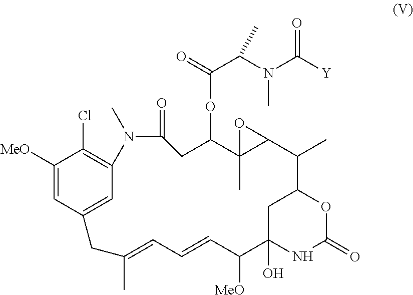

wherein Y represents (CR₇R₈)ₗ(CR₅R₆)ₘ(CR₃R₄)ₙCR₁R₂SZ,
wherein R₁ and R₂ are each independently CH₃, C₂H₅, linear alkyl, or alkenyl having from 1 to 10 carbon atoms, branched or cyclic alkyl or alkenyl having from 3 to 10 carbon atoms, phenyl, substituted phenyl or heterocyclic aromatic or heterocycloalkyl radical, and wherein R₂ also can be H,
wherein R₃, R₄, R₅, R₆, R₇, and R₈ are each independently H, CH₃, C₂H₅, linear alkyl or alkenyl having from 1 to 10 carbon atoms, branched or cyclic alkyl or alkenyl having from 3 to 10 carbon atoms, phenyl, substituted phenyl, or heterocyclic aromatic or heterocycloalkyl radical,
wherein l, m, and n are each independently an integer of from 1 to 5, and in addition n can be zero, and
wherein Z is H, SR or COR, wherein R is linear alkyl or alkenyl having from 1 to 10 carbon atoms, branched or cyclic alkyl or alkenyl having from 3 to 10 carbon atoms, or simple or substituted aryl or heterocyclic aromatic or heterocycloalkyl radical.

Preferred embodiments of formula (V) include compounds of formula (V) wherein (a) R₁ is H, R₂ is methyl, R₅, R₆, R₇, and R₈ are each H; 1 and m are each 1; n is 0; and Z is H, (b) R₁ and R₂ are methyl; R₅, R₆, R₇, R₈ are each H, 1 and m are 1; n is 0; and Z is H, (c) R₁ is H, R₂ is methyl, R₅, R₆, R₇, and R₈ are each H, 1 and m are each 1, n is 0, and Z is SCH₃, or (d) R₁ and R₂ are methyl, R₅, R₆, R₇, R₈ are each H, 1 and m are 1, n is 0, and Z is SCH₃.

Still further preferred maytansines include compounds represented by formula (VI-L), (VI-D), or (VI-D,L):

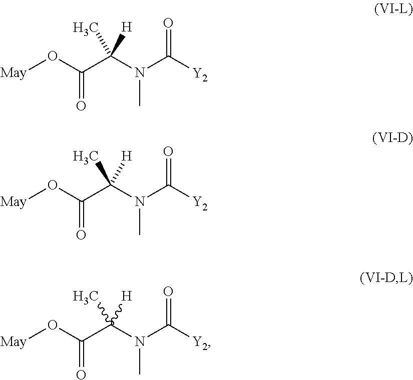

wherein Y₂ represents (CR₇R₈)ₗ(CR₅R₆)ₘ(CR₃R₄)ₙCR₁R₂SZ₂, wherein R₁ and R₂ are each independently CH₃, C₂H₅, linear alkyl or alkenyl having from 1 to 10 carbon atoms, branched or cyclic alkyl or alkenyl having from 3 to 10 carbon atoms, phenyl, substituted phenyl or heterocyclic aromatic or heterocycloalkyl radical, and wherein R₂ also can be H,
wherein R₃, R₄, R₅, R₆, R₇, and R₈ are each independently H, CH₃, C₂H₅, linear cyclic alkyl or alkenyl having from 1 to 10 carbon atoms, branched or cyclic alkyl or alkenyl having from 3 to 10 carbon atoms, phenyl, substituted phenyl or heterocyclic aromatic or heterocycloalkyl radical,
wherein l, m, and n are each independently an integer of from 1 to 5, and in addition n can be zero,
wherein Z₂ is SR or COR, wherein R is linear alkyl or alkenyl having from 1 to 10 carbon atoms, branched or cyclic alkyl or alkenyl having from 3 to 10 carbon atoms, or simple or substituted aryl or heterocyclic aromatic or heterocycloalkyl radical, and wherein May is a maytansinoid.

Additional preferred maytansines include compounds represented by formula (VII):

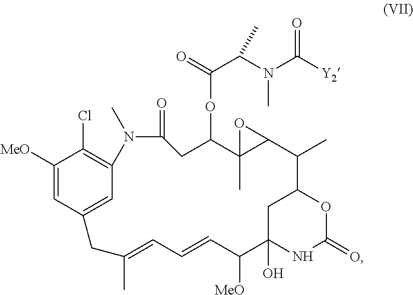

wherein Y_{2′} represents
(CR₇R₈)ₗ(CR₉═CR₁₀)ₚ(C≡C)_{q}Aₒ(CR₅R₆)ₘDᵤ(CR₁₁═CR₁₂)ᵣ(C≡C)ₛBₜ(CR₃R₄)CR₁R₂SZ₂, wherein R₁ and R₂ are each independently CH₃, C₂H₅, linear branched or alkyl or alkenyl having from 1 to 10 carbon atoms, cyclic alkyl or alkenyl having from 3 to 10 carbon atoms, phenyl, substituted phenyl or heterocyclic aromatic or heterocycloalkyl radical, and in addition R₂ can be H,
wherein A, B, and D each independently is cycloalkyl or cycloalkenyl having 3 to 10 carbon atoms, simple or substituted aryl, or heterocyclic aromatic or heterocycloalkyl radical, wherein R₃, R₄, R₅, R₆, R₇, R₈, R₉, R₁₀, R₁₁, and R₁₂ are each independently H, CH₃, C₂H₅, linear alkyl or alkenyl having from 1 to 10 carbon atoms, branched or cyclic alkyl or alkenyl having from 3 to 10 carbon atoms, phenyl, substituted phenyl or heterocyclic aromatic or heterocycloalkyl radical,
wherein l, m, n, o, p, q, r, s, and t are each independently zero or an integer of from 1 to 5, provided that at least two of l, m, n, o, p, q, r, s and t are not zero at any one time, and wherein Z₂ is SR or —COR, wherein R is linear alkyl or alkenyl having from 1 to 10 carbon atoms, branched or cyclic alkyl or alkenyl having from 3 to 10 carbon atoms, or simple or substituted aryl or heterocyclic aromatic or heterocycloalkyl radical.

Preferred embodiments of formula (VII) include compounds of formula (VII), wherein R₁ is H and R₂ is methyl.

In addition to maytansinoids, the cytotoxic agent used in the conjugate can be a taxane or derivative thereof. Taxanes are a family of compounds that includes paclitaxel (Taxol®), a cytotoxic natural product, and docetaxel (Taxotere®), a semi-synthetic derivative, which are both widely used in the treatment of cancer. Taxanes are mitotic spindle poisons that inhibit the depolymerization of tubulin, resulting in cell death. While docetaxel and paclitaxel are useful agents in the treatment of cancer, their antitumor activity is limited because of their non-specific toxicity towards normal cells. Further, compounds like paclitaxel and docetaxel themselves are not sufficiently potent to be used in conjugates of cell-binding agents.

A preferred taxane for use in the preparation of a cytotoxic conjugate is the taxane of formula (VIII):

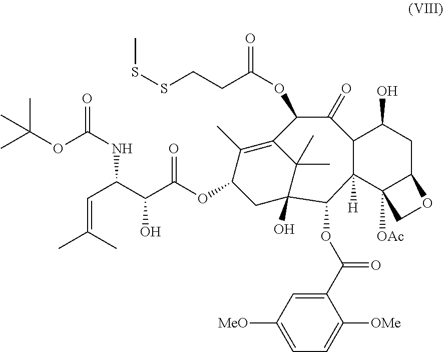 [SMILES: COc1ccc(OC)c(C(=O)O[C@H]2[C@H]3C(C(=O)[C@H](OC(=O)CCSSC)C4=C(C)[C@@H](OC(=O)[C@H](O)[C@H](C=C(C)C)NC(=O)OC(C)(C)C)C[C@]2(O)C4(C)C)[C@@H](O)C[C@H]2OC[C@@]32OC(C)=O)c1]

Methods for synthesizing taxanes that can be used in the context of the invention, along with methods for conjugating taxanes to cell-binding agents such as antibodies, are described in detail in U.S. Pat. Nos. 5,416,064, 5,475,092, 6,340,701, 6,372,738, 6,436,931, 6,596,757, 6,706,708, 6,716,821, and 7,390,898.

The cytotoxic agent also can be CC-1065 or a derivative thereof. CC-1065 is a potent anti-tumor antibiotic isolated from the culture broth of Streptomyces zelensis. CC-1065 is about 1000-fold more potent in vitro than commonly used anti-cancer drugs, such as doxorubicin, methotrexate, and vincristine (Bhuyan et al., Cancer Res., 42: 3532-3537 (1982)). CC-1065 and its analogs are disclosed in U.S. Pat. Nos. 5,585,499, 5,846,545, 6,340,701, and 6,372,738. The cytotoxic potency of CC-1065 has been correlated with its alkylating activity and its DNA-binding or DNA-intercalating activity. These two activities reside in separate parts of the molecule. In this respect, the alkylating activity is contained in the cyclopropapyrroloindole (CPI) subunit and the DNA-binding activity resides in the two pyrroloindole subunits of CC-1065.

Several CC-1065 analogs are known in the art and also can be used as the cytotoxic agent in the conjugate (see, e.g., Warpehoski et al., J. Med. Chem., 31: 590-603 (1988)). A series of CC-1065 analogs has been developed in which the CPI moiety is replaced by a cyclopropabenzindole (CBI) moiety (Boger et al., J. Org. Chem., 55: 5823-5833 (1990), and Boger et al., Bioorg. Med. Chem. Lett., 1: 115-120 (1991)). These CC-1065 analogs maintain the high in vitro potency of the parental drug, without causing delayed toxicity in mice. Like CC-1065, these compounds are alkylating agents that covalently bind to the minor groove of DNA to cause cell death.

The therapeutic efficacy of CC-1065 analogs can be greatly improved by changing the in vivo distribution through targeted delivery to a tumor site, resulting in lower toxicity to non-targeted tissues, and thus, lower systemic toxicity. To this end, conjugates of analogs and derivatives of CC-1065 with cell-binding agents that specifically target tumor cells have been generated (see, e.g., U.S. Pat. Nos. 5,475,092, 5,585,499, and 5,846,545). These conjugates typically display high target-specific cytotoxicity in vitro, and anti-tumor activity in human tumor xenograft models in mice (see, e.g., Chari et al., Cancer Res., 55: 4079-4084 (1995)).

Methods for synthesizing CC-1065 analogs are described in detail in U.S. Pat. Nos. 5,475,092, 5,585,499, 5,846,545, 6,534,660, 6,586,618, 6,756,397, and 7,329,760.

Drugs such as methotrexate, daunorubicin, doxorubicin, vincristine, vinblastine, melphalan, mitomycin C, chlorambucil, calicheamicin, tubulysin and tubulysin analogs, duocarmycin and duocarmycin analogs, dolastatin and dolastatin analogs also can be used as the cytotoxic agents of the invention. Doxarubicin and daunorubicin compounds (see, e.g., U.S. Pat. No. 6,630,579) can also be used as the cytotoxic agent.

The cell-binding agent cytotoxic agent conjugates may be prepared by in vitro methods. In order to link a cytotoxic agent to the antibody, a linking group is used. Suitable linking groups are well known in the art and include disulfide groups, acid labile groups, photolabile groups, peptidase labile groups, and esterase labile groups, as well as noncleavable linking groups.

In accordance with the invention, the cell-binding agent is modified by reacting a bifunctional crosslinking reagent with the cell-binding agent, thereby resulting in the covalent attachment of a linker molecule to the cell-binding agent. As used herein, a “bifunctional crosslinking reagent” refers to a reagent that possesses two reactive groups; one of which is capable of reacting with a cell-binding agent, while the other one is capable of reacting with the cytotoxic agent to link the cell-binding agent with the cytotoxic agent, thereby forming a conjugate.

Any suitable bifunctional crosslinking reagent can be used in connection with the invention, so long as the linker reagent provides for retention of the therapeutic, e.g., cytotoxicity, and targeting characteristics of the cytotoxic agent and the cell-binding agent, respectively, while providing an acceptable toxicity profile. Preferably, the linker molecule joins the cytotoxic agent to the cell-binding agent through chemical bonds (as described above), such that the cytotoxic agent and the cell-binding agent are chemically coupled (e.g., covalently bonded) to each other.

In one embodiment, the cell binding agent is chemically coupled to the cytotoxic agent via chemical bonds selected from the group consisting of disulfide bonds, acid labile bonds, photolabile bonds, peptidase labile bonds, and esterase labile bonds.

In one embodiment, the bifunctional crosslinking reagent comprises non-cleavable linkers. A non-cleavable linker is any chemical moiety that is capable of linking a cytotoxic agent, such as a maytansinoid, a taxane, or a CC-1065 analog, to a cell-binding agent in a stable, covalent manner. Thus, non-cleavable linkers are substantially resistant to acid-induced cleavage, light-induced cleavage, peptidase-induced cleavage, esterase-induced cleavage, and disulfide bond cleavage, at conditions under which the cytotoxic agent or the cell-binding agent remains active.

Suitable crosslinking reagents that form non-cleavable linkers between a cytotoxic agent and the cell-binding agent are well known in the art. In one embodiment, the cytotoxic agent is chemically coupled to the cell-binding agent through a thioether bond. In another embodiment, the cytotoxic agent is linked to the cell-binding agent through an amide bond. Examples of non-cleavable linkers include linkers having a maleimido-based moeity or a haloacetyl-based moiety for reaction with the cytotoxic agent. Such bifunctional crosslinking agents are well known in the art (see U.S. Patent Application Publication Nos. 20100129314, 20090274713, 20080050310, 20050169933, and Pierce Biotechnology Inc. P.O. Box 117, Rockland, Ill. 61105, USA) and include, but not limited to, N-succinimidyl 4-(maleimidomethyl)cyclohexanecarboxylate (SMCC), N-succinimidyl-4-(N-maleimidomethyl)-cyclohexane-1-carboxy-(6-amidocaproate), which is a “long chain” analog of SMCC (LC-SMCC), κ-maleimidoundecanoic acid N-succinimidyl ester (KMUA), γ-maleimidobutyric acid N-succinimidyl ester (GMBS), ε-maleimidocaproic acid N-hydroxysuccinimide ester (EMCS), m-maleimidobenzoyl-N-hydroxysuccinimide ester (MBS), N-(α-maleimidoacetoxy)-succinimide ester (AMAS), succinimidyl-6-((3-maleimidopropionamido)hexanoate (SMPH), N-succinimidyl 4-(p-maleimidophenyl)-butyrate (SMPB), and N-(p-maleimidophenyl)isocyanate (PMPI). Cross-linking reagents comprising a haloacetyl-based moiety include N-succinimidyl-4-(iodoacetyl)-aminobenzoate (SIAB), N-succinimidyl iodoacetate (SIA), N-succinimidyl bromoacetate (SBA), N-succinimidyl 3-(bromoacetamido)propionate (SBAP), bis-maleimidopolyethyleneglycol (BMPEO), BM(PEO)₂, BM(PEO)₃, N-(β-maleimidopropyloxy)succinimide ester (BMPS), 5-maleimidovaleric acid NHS, HBVS, 4-(4-N-maleimidophenyl)-butyric acid hydrazideHC1 (MPBH), Succinimidyl-(4-vinylsulfonyl)benzoate (SVSB), dithiobis-maleimidoethane (DTME), 1,4-bis-maleimidobutane (BMB), 1,4 bismaleimidyl-2,3-dihydroxybutane (BMDB), bis-maleimidohexane (BMH), bis-maleimidoethane (BMOE), sulfosuccinimidyl 4-(N-maleimido-methyl)cyclohexane-1-carboxylate (sulfo-SMCC), sulfosuccinimidyl(4-iodo-acetyl)aminobenzoate (sulfo-SIAB), m-Maleimidobenzoyl-N-hydroxysulfosuccinimide ester (sulfo-MBS), N-(γ-maleimidobutryloxy)sulfosuccinimde ester (sulfo-GMBS), N-(8-maleimidocaproyloxy)sulfosuccimido ester (sulfo-EMCS), N-(K-maleimidoundecanoyloxy)sulfosuccinimide ester (sulfo-KMUS), sulfosuccinimidyl 4-(p-maleimidophenyl)butyrate (sulfo-SMPB), CX1-1, sulfo-Mal, and PEG-Mal. Preferably, the bifunctional crosslinking reagent is SMCC.

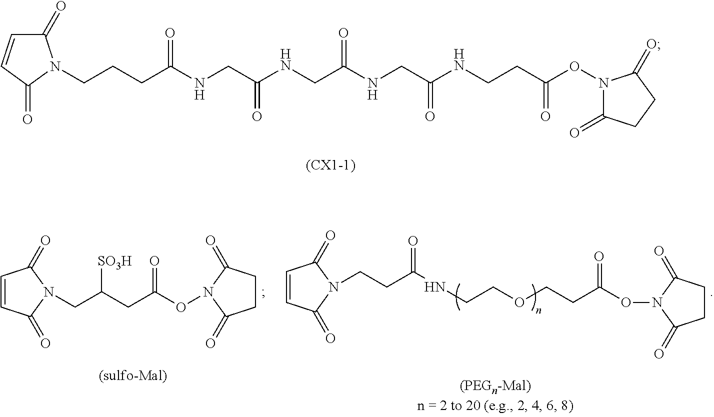

In one embodiment, the linking reagent is a cleavable linker. Examples of suitable cleavable linkers include disulfide containing linkers, acid labile linkers, photolabile linkers, peptidase labile linkers, and esterase labile linkers. Disulfide containing linkers are linkers cleavable through disulfide exchange, which can occur under physiological conditions. Acid labile linkers are linkers cleavable at acid pH. For example, certain intracellular compartments, such as endosomes and lysosomes, have an acidic pH (pH 4-5), and provide conditions suitable to cleave acid labile linkers. Photo labile linkers are useful at the body surface and in many body cavities that are accessible to light. Furthermore, infrared light can penetrate tissue. Peptidase labile linkers can be used to cleave certain peptides inside or outside cells (see e.g., Trouet et al., Proc. Natl. Acad. Sci. USA, 79: 626-629 (1982), and Umemoto et al., Int. J. Cancer, 43: 677-684 (1989)). In one embodiment, the cleavable linker is cleaved under mild conditions, i.e., conditions within a cell under which the activity of the cytotoxic agent is not affected.

In one embodiment, the cytotoxic agent is linked to a cell-binding agent through a disulfide bond. The linker molecule comprises a reactive chemical group that can react with the cell-binding agent. In one embodiment, the bifunctional crosslinking reagent comprises a reactive moiety that can form an amide bond with a lysine residue of the cell-binding agent. Examples of reactive moieties that can form an amide bond with a lysine residue of a cell-binding agent include carboxylic acid moieties and reactive ester moieties, such as N-succinimidyl ester, N-sulfosuccinimidyl ester, nitrophenyl (e.g., 2 or 4-nitrophenyl) ester, dinitrophenyl (e.g., 2,4-dinitrophenyl) ester, sulfo-tetraflurophenyl (e.g., 4-sulfo-2,3,5,6-tetrafluorophenyl) ester, and pentafluorophenyl ester.

Preferred reactive chemical groups for reaction with the cell-binding agent are N-succinimidyl esters and N-sulfosuccinimidyl esters. Additionally the linker molecule comprises a reactive chemical group, preferably a dithiopyridyl group, that can react with the cytotoxic agent to form a disulfide bond. Bifunctional crosslinking reagents that enable the linkage of the cell-binding agent with the cytotoxic agent via disulfide bonds are known in the art and include, for example, N-succinimidyl 3-(2-pyridyldithio)propionate (SPDP) (see, e.g., Carlsson et al., Biochem. J., 173: 723-737 (1978)), N-succinimidyl 4-(2-pyridyldithio)butanoate (SPDB) (see, e.g., U.S. Pat. No. 4,563,304), N-succinimidyl 4-(2-pyridyldithio)pentanoate (SPP) (see, e.g., CAS Registry number 341498-08-6), and N-succinimidyl-4-(2-pyridyldithio)2-sulfo butanoate (sulfo-SPDB) (see, e.g., U.S. Patent Application Publication No. 20090274713). Other bifunctional crosslinking reagents that can be used to introduce disulfide groups are known in the art and are described in U.S. Pat. Nos. 6,913,748, 6,716,821 and U.S. Patent Application Publication Nos. 20090274713 and 20100129314, all of which are incorporated herein in its entirety by reference.

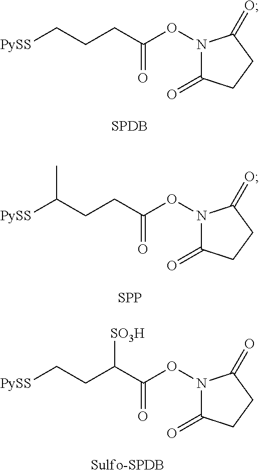

Other crosslinking reagents lacking a sulfur atom that form non-cleavable linkers can also be used in the inventive method. Such linkers can be derived from dicarboxylic acid based moieties. Suitable dicarboxylic acid based moieties include, but are not limited to, α,ω-dicarboxylic acids of the general formula (IX):

HOOC—X₁—Yₙ—Zₘ—COOH (IX),

wherein X is a linear or branched alkyl, alkenyl, or alkynyl group having 2 to 20 carbon atoms, Y is a cycloalkyl or cycloalkenyl group bearing 3 to 10 carbon atoms, Z is a substituted or unsubstituted aromatic group bearing 6 to 10 carbon atoms, or a substituted or unsubstituted heterocyclic group wherein the hetero atom is selected from N, O or S, and wherein l, m, and n are each 0 or 1, provided that l, m, and n are all not zero at the same time.

Many of the non-cleavable linkers disclosed herein are described in detail in U.S. Patent Application Publication No. 20050169933 A1.

Final purified cell-binding agent cytotoxic agent conjugates produced by the inventive process comprise a cytotoxic agent, a bifunctional crosslinking agent, and a cell-binding agent. In a preferred embodiment of the invention, the cytotoxic agent is DM1, the bifunctional crosslinking agent is SMCC, and the cell-binding agent is huCD37-3 antibody. In another preferred embodiment of the invention, the cytotoxic agent is DM1, the bifunctional crosslinking agent is SMCC, and the cell-binding agent is EGFR-7R antibody. In a preferred embodiment of the invention, the cytotoxic agent is DM1, the bifunctional crosslinking agent is SMCC, and the cell-binding agent is an anti-EFGRvIII antibody. In a preferred embodiment of the invention, the cytotoxic agent is DM1, the bifunctional crosslinking agent is SMCC, and the cell-binding agent is an anti-CD27L antibody. In a preferred embodiment of the invention, the cytotoxic agent is DM1, the bifunctional crosslinking agent is SMCC, and the cell-binding agent is trastuzumab.

The following examples further illustrate the invention but, of course, should not be construed as in any way limiting its scope.

### Example 1

This example demonstrates a process for preparing a purified cell-binding agent cytotoxic agent conjugate comprising subjecting a mixture comprising a cell-binding agent cytotoxic agent conjugate and one or more impurities to an ion exchange chromatography membrane to remove at least a portion of the impurities from the mixture, thereby providing a purified cell-binding agent cytotoxic agent conjugate.

An antibody (Ab) was buffered in 50 mM KPi, 2 mM EDTA at pH 7.5. The buffered Ab was mixed with 10% (vv) DMA, 1.1 molar excesses of DM1 relative to linker (SMCC) and 8.6 molar equivalents of SMCC per moles of Ab. Reactions proceeded for 24 hrs at 15° C. Then, half of the reaction mixture was applied to Sartobid IEX SingleSep (Q membrane), and the other half was used as control. The mixtures (before and after Q membrane) were analyzed. The results are shown in Table 1 below.

TABLE 1

| | Control | Q filtrate |
|---|---|---|
| High Molecular Weight Species | 0.4% | 0 |
| Conjugate Dimer | 2.1% | 0.6% |
| Conjugate Monomer | 97.0% | 99.0% |
| Low Molecular Weight Species | 0.5% | 0.4% |
| DM1-DM1* | 15.8% | 6.1% |
| DM1-MCC-DM1* | 12.3% | 2.2% |

*percentage relative to total free DM1 species

As shown in Table 1, the conjugate filtered through a Q membrane has a lower level of high molecular weight species (both higher order aggregates and conjugate dimers) and a higher level of conjugate monomer. In addition, DM1-DM1 dimers and DM1-MCC-DM1 dimers are efficiently removed by the Q membrane.

The results of the experiments reflected in this example demonstrate that an ion exchange chromatography membrane, specifically a Q membrane, can be used to remove at least a portion of the impurities from a mixture comprising a cell-binding agent cytotoxic agent conjugate. In particular, the Q membrane efficiently removed cytotoxic agent dimers DM1-DM1 and DM1-MCC-DM1 from a mixture comprising a cell-binding agent cytotoxic agent conjugate and one or more impurities.

### Example 2

This example demonstrates a process for preparing a purified cell-binding agent cytotoxic agent conjugate comprising subjecting a mixture comprising a cell-binding agent cytotoxic agent conjugate and one or more impurities to an ion exchange chromatography membrane to remove at least a portion of the impurities from the mixture, thereby providing a purified cell-binding agent cytotoxic agent conjugate.

An antibody-CX1-1-DM1 conjugate was prepared as described in Example 1. The mixtures (before and after Q membrane) were analyzed. The results are shown in Table 2 below.

TABLE 2

| | High | | | Low |
|---|---|---|---|---|
| | Molecular | | | Molecular |
| | Weight | Conjugate | Conjugate | Weight |
| | Species | dimers | Monomer | Species |
| Control | 0.47% | 2.96% | 96.5% | 0.08% |
| Q filter | 0.0% | 1.9% | 98.1% | 0.1% |

As shown in Table 2, the conjugate filtered through a Q membrane has a lower level of high molecular weight species (both higher order aggregates and conjugate dimers) and a higher level of conjugate monomer.

The results of the experiments reflected in this example demonstrate that an ion exchange chromatography membrane, specifically a Q membrane, can be used to remove at least a portion of the impurities from a mixture comprising a cell-binding agent cytotoxic agent conjugate. In particular, the Q membrane efficiently removed high molecular weight species (e.g., aggregates of conjugates) from a mixture comprising a cell-binding agent cytotoxic agent conjugate and one or more impurities.

### Example 3

This example demonstrates a process for preparing a purified cell-binding agent cytotoxic agent conjugate comprising subjecting a mixture comprising a cell-binding agent cytotoxic agent conjugate and one or more impurities to an ion exchange chromatography membrane to remove at least a portion of the impurities from the mixture, thereby providing a purified cell-binding agent cytotoxic agent conjugate.

An antibody (Ab) was buffer-exchanged into 15 mM KPi, 2 mM EDTA, at pH 7.6. The buffer-exchanged Ab was mixed with 5.3 molar of DM4 and 4.4 molar of Sulfo-SPDB per moles of Ab in 8% (vv) DMA. Reactions proceeded for 20 hours at 20° C. The conjugation mixture was subjected to tangential flow filtration for buffer exchange and purification of the antibody conjugate. 1 gram of purified Ab-Sulfo-SPDB-DM4 conjugate was applied to Pall's Mustang Q Coin (Pall Cat # MSTA18Q16). Fractions were collected and analyzed for free maytansinoid species. The results are shown in Table 3 below.

TABLE 3

| | Concentration (ng/mL) | | | |
|---|---|---|---|---|
| | DM4-Hydrolyzed- | | | |
| Fractions | Sulfo-SPDB | DM4 | DM4-SPy | DM4-Sulfo-TBA |
| Pre-Q | 18 | 11 | 195 | 1918 |
| Membrane | | | | |
| Post-Q | 0 | 7 | 83 | 242 |
| Membrane | | | | |

As shown in Table 3, the antibody conjugate filtered through the Q membrane has a lower level of each identified free maytansinoid species as compared to the antibody conjugate that was not filtered through the Q membrane.

The results of the experiments reflected in this example demonstrate that an ion exchange chromatography membrane, specifically a Q membrane, can be used to remove free cytotoxic agent impurities from a mixture comprising a cell-binding agent cytotoxic agent conjugate. In particular, the Q membrane reduced the levels of DM4-hydrolyzed-sulfo-SPDB, DM4, DM4-SPy, and DM4-sulfo-TBA in a mixture comprising a cell-binding agent cytotoxic agent conjugate and one or more impurities.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A process for preparing a purified cell-binding agent cytotoxic agent conjugate comprising subjecting a mixture comprising a cell-binding agent cytotoxic agent conjugate and one or more impurities to an ion exchange chromatography membrane to remove at least a portion of the impurities from the mixture, thereby providing a purified cell-binding agent cytotoxic agent conjugate.

2. The process of claim 1, wherein the process is sequentially repeated two, three, or four times.

3. The process of claim 1, wherein the process comprises:
(a) contacting a cell-binding agent with a cytotoxic agent to form a first mixture comprising the cell-binding agent and the cytotoxic agent, then contacting the first mixture with a bifunctional crosslinking reagent comprising a linker, in a solution having a pH of about 4 to about 9, to provide a second mixture comprising the cell-binding agent cytotoxic agent conjugate comprising the cell-binding agent chemically coupled through the linker to the cytotoxic agent and one or more impurities;
(b) subjecting the second mixture to an ion exchange chromatography membrane to remove at least a portion of the impurities from the mixture, thereby providing a purified second mixture of the cell-binding agent cytotoxic agent conjugate; and
(c) subjecting the purified second mixture after step (b) to tangential flow filtration, selective precipitation, non-adsorptive chromatography, adsorptive filtration, adsorptive chromatography, or a combination thereof, to further purify the cell-binding agent-cytotoxic agent conjugate from the impurities and thereby prepare a purified third mixture of the cell-binding agent-cytotoxic agent conjugate, wherein the purified third mixture comprises a reduced amount of the impurities as compared to the purified second mixture.

4. The process of claim 3, wherein step (b) is sequentially repeated two, three, or four times prior to step (c).

5. The process of claim 3, wherein adsorptive chromatography is utilized in step (c).

6. The process of claim 3, wherein the adsorptive chromatography is selected from the group consisting of hydroxyapatite chromatography, hydrophobic charge induction chromatography (HCIC), hydrophobic interaction chromatography (HIC), ion exchange chromatography, mixed mode ion exchange chromatography, immobilized metal affinity chromatography (IMAC), dye ligand chromatography, affinity chromatography, reversed phase chromatography, and combinations thereof.

7. The process of claim 6, wherein the adsorptive chromatography is ion exchange chromatography.

8. The process of claim 7, wherein the ion exchange chromatography is ceramic hydroxyapatite (CHT) chromatography.

9. The process of claim 3, wherein tangential flow filtration is utilized in step (c).

10. The process of claim 3, wherein the contacting in step (a) is effected by providing the cell-binding agent in a reaction vessel, adding the cytotoxic agent to the reaction vessel to form the first mixture comprising the cell-binding agent and the cytotoxic agent, and then adding the bifunctional crosslinking reagent to the first mixture.

11. The process of claim 3, further comprising holding the mixture between steps a-b or steps b-c to release the unstably bound linkers from the cell-binding agent.

12. The process of claim 11, wherein the mixture is held for about 20 hours at a temperature of about 2° C. to about 8° C.

13. The process of claim 3, further comprising quenching the second mixture between steps (a)-(b) to quench any unreacted cytotoxic agent and/or unreacted bifunctional crosslinking reagent.

14. The process of claim 13, wherein the mixture is quenched by contacting the second mixture with a quenching reagent that reacts with the free cytotoxic agent.

15. The process of claim 14, wherein the quenching reagent is selected from the group consisting of 4-maleimidobutyric acid, 3-maleimidopropionic acid, N-ethylmaleimide, iodoacetamide, and iodoacetamidopropionic acid.

16. The process of claim 3, wherein the process comprises
(a) contacting a cell-binding agent with a cytotoxic agent to form a first mixture comprising the cell-binding agent and the cytotoxic agent, then contacting the first mixture with a bifunctional crosslinking reagent comprising a linker, in a solution having a pH of about 4 to about 9, to provide a second mixture comprising the cell-binding agent cytotoxic agent conjugate comprising the cell-binding agent chemically coupled through the linker to the cytotoxic agent and one or more impurities;
(b) subjecting the second mixture to an ion exchange chromatography membrane to remove at least a portion of the impurities from the mixture, thereby providing a purified second mixture of the cell-binding agent cytotoxic agent conjugate;
(c) quenching the purified second mixture after step (b) to quench any unreacted cytotoxic agent and/or unreacted bifunctional crosslinking reagent;
(d) subjecting the quenched mixture after step (c) to an ion exchange chromatography membrane to remove at least a portion of the impurities from the mixture, thereby providing a purified third mixture of the cell-binding agent cytotoxic agent conjugate;
(e) holding the purified third mixture to release the unstably bound linkers from the cell-binding agent;
(f) subjecting the purified third mixture after step (c) to an ion exchange chromatography membrane to remove at least a portion of the impurities from the mixture, thereby providing a purified fourth mixture of the cell-binding agent cytotoxic agent conjugate; and
(g) subjecting the purified fourth mixture after step (f) to tangential flow filtration, selective precipitation, non-adsorptive chromatography, adsorptive filtration, adsorptive chromatography, or a combination thereof, to further purify the cell-binding agent-cytotoxic agent conjugate from the impurities and thereby prepare a purified third mixture of the cell-binding agent-cytotoxic agent conjugate, wherein the purified third mixture comprises a reduced amount of the impurities as compared to the purified second mixture.

17. The process of claim 16, wherein tangential flow filtration is utilized in step (g).

18. The process of claim 1, wherein the process comprises:
(a) contacting a cell-binding agent with a bifunctional crosslinking reagent to covalently attach a linker to the cell-binding agent and thereby prepare a first mixture comprising cell-binding agents having linkers bound thereto,
(b) subjecting the first mixture to tangential flow filtration, selective precipitation, non-adsorptive chromatography, adsorptive filtration, adsorptive chromatography, or a combination thereof and thereby prepare a purified first mixture of cell-binding agents having linkers bound thereto,
(c) conjugating a cytotoxic agent to the cell-binding agents having linkers bound thereto in the purified first mixture by reacting the cell-binding agents having linkers bound thereto with a cytotoxic agent in a solution having a pH of about 4 to about 9 to prepare a second mixture comprising the cell-binding agent-cytotoxic agent conjugate comprising the cell-binding agent chemically coupled to the cytotoxic agent through the linker and one or more impurities,
(d) subjecting the second mixture to an ion exchange chromatography membrane to remove at least a portion of the impurities, thereby providing a purified second mixture of the cell-binding agent cytotoxic agent conjugate; and
(e) subjecting the purified second mixture after step (d) to tangential flow filtration, selective precipitation, non-adsorptive chromatography, adsorptive filtration, adsorptive chromatography, or a combination thereof, to further purify the cell-binding agent-cytotoxic agent conjugate from the impurities and thereby prepare a purified third mixture of the cell-binding agent-cytotoxic agent conjugate, wherein the purified third mixture comprises a reduced amount of the impurities as compared to the purified second mixture.

19. The process of claim 18, wherein step (d) is sequentially repeated two, three, or four times prior to step (e).

20. The process of claim 18, wherein adsorptive chromatography is utilized in steps (b) and (d).

21. The process of claim 18, wherein tangential flow filtration is utilized in step (b) and adsorptive chromatography is utilized in step (d).

22. The process of claim 18, wherein adsorptive chromatography is utilized in step (b) and tangential flow filtration is utilized in step (d).

23. The process of claim 18, wherein the adsorptive chromatography is selected from the group consisting of hydroxyapatite chromatography, hydrophobic charge induction chromatography (HCIC), hydrophobic interaction chromatography (HIC), ion exchange chromatography, mixed mode ion exchange chromatography, immobilized metal affinity chromatography (IMAC), dye ligand chromatography, affinity chromatography, reversed phase chromatography, and combinations thereof.

24. The process of claim 23, wherein the adsorptive chromatography is ion-exchange chromatography.

25. The process of claim 24, wherein the ion-exchange chromatography is ceramic hydroxyapatite (CHT) chromatography.

26. The process of claim 18, wherein tangential flow filtration is utilized in steps (b) and (d).

27. The process of claim 18, wherein non-adsorptive chromatography is utilized in steps (b) and (d).

28. The process of claim 18, wherein the solution in step (c) comprises sucrose.

29. The process of claim 18, wherein the solution in step (c) comprises a buffering agent selected from the group consisting of a citrate buffer, an acetate buffer, a succinate buffer, and a phosphate buffer.

30. The process of claim 18, wherein the solution in step (c) comprises a buffering agent selected from the group consisting of HEPPSO (N-(2-Hydroxyethyl)piperazine-N′-(2-hydroxypropanesulfonic acid)), POPSO (Piperazine-1,4-bis-(2-hydroxy-propane-sulfonic acid) dehydrate), HEPES (4-(2-hydroxyethyl)piperazine-1-ethanesulfonic acid), HEPPS (EPPS) (4-(2-hydroxyethyl)piperazine-1-propanesulfonic acid), TES (N-[tris(hydroxymethyl)methyl]-2-aminoethanesulfonic acid), and a combination thereof.

31. The process of claim 18, further comprising
(f) holding the mixture between at least one of steps a-b, steps b-c, steps c-d, and steps d-e to release the unstably bound linkers from the cell-binding agent.

32. The process of claim 1, wherein the process comprises:
(a) contacting a cell-binding agent with a bifunctional crosslinking reagent to covalently attach a linker to the cell-binding agent and thereby prepare a first mixture comprising cell-binding agents having linkers bound thereto,
(b) conjugating a cytotoxic agent to the cell-binding agents having linkers bound thereto in the first mixture by reacting the cell-binding agents having linkers bound thereto with a cytotoxic agent to prepare a second mixture comprising the cell-binding agent-cytotoxic agent conjugate comprising the cell-binding agent chemically coupled through the linker to the cytotoxic agent and one or more impurities,
(c) subjecting the second mixture to an ion exchange chromatography membrane to remove at least a portion of the impurities, thereby providing a purified second mixture of the cell-binding agent cytotoxic agent conjugate; and
(d) subjecting the purified second mixture after step (c) to tangential flow filtration, selective precipitation, non-adsorptive chromatography, adsorptive filtration, adsorptive chromatography, or a combination thereof, to further purify the cell-binding agent-cytotoxic agent conjugate from the impurities and thereby prepare a purified third mixture of the cell-binding agent-cytotoxic agent conjugate, wherein the purified third mixture comprises a reduced amount of the impurities as compared to the purified second mixture.

33. The process of claim 32, wherein the first mixture is not subjected to purification between steps (a) and (b).

34. The process of claim 32, wherein step (c) is sequentially repeated two, three, or four times prior to step (d).

35. The process of claim 32, wherein adsorptive chromatography is utilized in step (d).

36. The process of claim 35, wherein the adsorptive chromatography is selected from the group consisting of hydroxyapatite chromatography, hydrophobic charge induction chromatography (HCIC), hydrophobic interaction chromatography (HIC), ion exchange chromatography, mixed mode ion exchange chromatography, immobilized metal affinity chromatography (IMAC), dye ligand chromatography, affinity chromatography, reversed phase chromatography, and combinations thereof.

37. The process of claim 36, wherein the adsorptive chromatography is ion-exchange chromatography.

38. The process of claim 37, wherein the ion-exchange chromatography is ceramic hydroxyapatite (CHT) chromatography.

39. The process of claim 32, wherein tangential flow filtration is utilized in step (d).

40. The process of claim 32, wherein
non-adsorptive chromatography is utilized in step (d).

41. The process of claim 32, wherein the solution in step (b) comprises sucrose.

42. The process of claim 32, wherein the solution in step (b) comprises a buffering agent selected from the group consisting of a citrate buffer, an acetate buffer, a succinate buffer, and a phosphate buffer.

43. The process of claim 32, wherein the solution in step (b) comprises a buffering agent selected from the group consisting of HEPPSO (N-(2-Hydroxyethyl)piperazine-N′-(2-hydroxypropanesulfonic acid)), POPSO (Piperazine-1,4-bis-(2-hydroxy-propane-sulfonic acid) dehydrate), HEPES (4-(2-hydroxyethyl)piperazine-1-ethanesulfonic acid), HEPPS (EPPS) (4-(2-hydroxyethyl)piperazine-1-propanesulfonic acid), TES (N-[tris(hydroxymethyl)methyl]-2-aminoethanesulfonic acid), and a combination thereof.

44. The process of claim 32, further comprising
(e) holding the mixture between at least one of steps a-b, steps b-c, and steps c-d to release the unstably bound linkers from the cell-binding agent.

45. The process of claim 1, wherein the one or more impurities are selected from the group of cytotoxic agent dimers, aggregates of the cell-binding agent cytotoxic agent conjugate, free cytotoxic agent, unconjugated linker, and mixtures thereof.

46. The process of claim 45, wherein the mixture comprises cytotoxic agent dimers as an impurity, and some portion of the cytotoxic agent dimers is removed from the mixture to provide the purified cell-binding agent cytotoxic agent conjugate.

47. The process of claim 46, wherein the cytotoxic agent dimer comprises DM1-DM1.

48. The process of claim 46, wherein the cytotoxic agent dimer comprises DM1-MCC-DM1.

49. The process of claim 46, wherein the cytotoxic agent dimer comprises DM1-DM1 and DM1-MCC-DM1.

50. The process of claim 45, wherein the mixture comprises aggregates of the cell-binding agent cytotoxic agent conjugate as an impurity, and some portion of the aggregates of the cell-binding agent cytotoxic agent conjugate is removed from the mixture to provide the purified cell-binding agent cytotoxic agent conjugate.

51. The process of claim 45, wherein the mixture comprises free cytotoxic agent as an impurity, and some portion of the free cytotoxic agent is removed from the mixture to provide the purified cell-binding agent cytotoxic agent conjugate.

52. The process of claim 45, wherein the mixture comprises unconjugated linker as an impurity, and some portion of the unconjugated linker is removed from the mixture to provide the purified cell-binding agent cytotoxic agent conjugate.

53. The process of claim 1, wherein the pH of the mixture that is subjected to the ion exchange chromatography membrane is about 4 to about 9.

54. The process of claim 53, wherein the pH of the mixture is about 7 to about 8.

55. The process of claim 54, wherein the pH of the mixture is about 7.3 to about 7.7.

56. The process of claim 55, wherein the pH of the mixture is about 7.5.

57. The process of claim 53, wherein the pH of the mixture is about 4.5 to about 5.5.

58. The process of claim 57, wherein the pH of the mixture is about 4.8 or about 5.

59. The process of claim 1, wherein at least 50% of the one or more impurities are removed from the mixture.

60. The process of claim 1, wherein at least 75% of the one or more impurities are removed from the mixture.

61. The process of claim 1, wherein at least 90% of the one or more impurities are removed from the mixture.

62. The process of claim 1, wherein the ion exchange chromatography membrane is an anion exchange membrane.

63. The process of claim 62, wherein the anion exchange membrane is a Q membrane.

64. The process of claim 1, wherein the ion exchange chromatography membrane is a cation exchange membrane.

65. The process of claim 64, wherein the cation exchange membrane is a S membrane.

66. The process of claim 1, wherein the ion exchange chromatography membrane is an endotoxin removal exchange membrane.

67. The process of claim 3, wherein the contacting in step (a) occurs in a solution having a pH of about 7 to about 9.

68. The process of claim 3, wherein the solution in step (a) comprises a buffering agent selected from the group consisting of a citrate buffer, an acetate buffer, a succinate buffer, and a phosphate buffer.

69. The process of claim 3, wherein the solution in step (a) comprises a buffering agent selected from the group consisting of HEPPSO (N-(2-Hydroxyethyl)piperazine-N′-(2-hydroxypropanesulfonic acid)), POPSO (Piperazine-1,4-bis-(2-hydroxy-propane-sulfonic acid) dehydrate), HEPES (4-(2-hydroxyethyl)piperazine-1-ethanesulfonic acid), HEPPS (EPPS) (4-(2-hydroxyethyl)piperazine-1-propanesulfonic acid), TES (N-[tris(hydroxymethyl)methyl]-2-aminoethanesulfonic acid), and a combination thereof.

70. The process of claim 3, wherein the contacting in step (a) occurs at a temperature of about 16° C. to about 24° C.

71. The process of claim 3, wherein the contacting in step (a) occurs at a temperature of about 0° C. to about 15° C.

72. The process of claim 3, wherein the bifunctional crosslinking reagent is an acid labile linker, a disulfide containing linker, a photolabile linker, a peptidase labile linker, or an esterase labile linker.

73. The process of claim 3, wherein the bifunctional crosslinking reagent is a disulfide-containing cleavable linker.

74. The process of claim 3, wherein the bifunctional crosslinking reagent is a non-cleavable linker.

75. The process of claim 3, wherein the bifunctional crosslinking reagent comprises an N-succinimidyl ester moiety, an N-sulfosuccinimidyl ester moiety, a maleimido-based moiety, or a haloacetyl-based moiety.

76. The process of claim 73, wherein the bifunctional crosslinking reagent is selected from the group consisting of N-succinimidyl 3-(2-pyridyldithio)propionate (SPDP), N-succinimidyl 4-(2-pyridyldithio)butanoate (SPDB), N-succinimidyl 4-(2-pyridyldithio)pentanoate (SPP), and N-succinimidyl-4-(2-pyridyldithio)2-sulfo butanoate (sulfo-SPDB).

77. The process of claim 74, wherein the bifunctional crosslinking reagent is selected from the group consisting of N-succinimidyl 4-(maleimidomethyl)cyclohexanecarboxylate (SMCC), N-succinimidyl-4-(N-maleimidomethyl)-cyclohexane-1-carboxy-(6-amidocaproate) (LC-SMCC), K-maleimidoundecanoic acid N-succinimidyl ester (KMUA), γ-maleimidobutyric acid N-succinimidyl ester (GMBS), β-maleimidopropyloxy-succinimidyl ester (BMPS), ε-maleimidocaproic acid N-hydroxysuccinimide ester (EMCS), m-maleimidobenzoyl-N-hydroxysuccinimide ester (MBS), N-(α-maleimidoacetoxy)-succinimide ester (AMAS), succinimidyl-6-(β-maleimidopropionamido)hexanoate (SMPH), N-succinimidyl 4-(p-maleimidophenyl)-butyrate (SMPB), and N-(p-maleimidophenyl)isocyanate (PMPI), sulfo-Mal, PEG₄-Mal and CX1-1.

78. The process of claim 1, wherein the cell-binding agent is selected from the group consisting of antibodies, interferons, interleukin 2 (IL-2), interleukin 3 (IL-3), interleukin 4 (IL-4), interleukin 6 (IL-6), insulin, EGF, TGF-α, FGF, G-CSF, VEGF, MCSF, GM-CSF, and transferrin.

79. The process of claim 78, wherein the cell-binding agent is an antibody.

80. The process of claim 79, wherein the antibody is a monoclonal antibody.

81. The process of claim 80, wherein the antibody is a humanized monoclonal antibody.

82. The process of claim 78, wherein the cell-binding agent is an antibody selected from the group consisting of huB4, huC242, trastuzumab, bivatuzumab, sibrotuzumab, huDS6, rituximab, anti-CD33 antibody, anti-CD27L antibody, anti-Her2 antibody, anti-EGFR antibody, anti-EGFRvIII antibody, Cripto, anti-CD138 antibody, anti-CD38 antibody, anti-EphA2 antibody, integrin targeting antibody, anti-CD37 antibody, anti-folate receptor antibody, anti-Her3 antibody, B-B4 antibody and anti-IGFIR antibody.

83. The process of claim 1, wherein the cytotoxic agent is selected from the group consisting of maytansinoids, taxanes, and CC1065.

84. The process of claim 83, wherein the cytotoxic agent is a maytansinoid.

85. The process of claim 84, wherein the maytansinoid comprises a thiol group.

86. The process of claim 85, wherein the maytansinoid is N^{2′}-deacetyl-N^{2′}-(3-mercapto-1-oxopropyl)-maytansine (DM1) or N^{2′}-de acetyl-N^{2′}-(4-methyl-4-mercapto-1-oxopentyl)-maytansine (DM4).

87. The process of claim 1, wherein the cytotoxic agent is DM1, the bifunctional crosslinking agent is SMCC, and the cell-binding agent is huCD37-3 antibody.

88. The process of claim 1, wherein the cytotoxic agent is DM1, the bifunctional crosslinking agent is SMCC, and the cell-binding agent is EGFR-7R antibody.

89. The process of claim 1, wherein the cytotoxic agent is DM1, the bifunctional crosslinking agent is SMCC, and the cell-binding agent is an anti-EFGRvIII antibody.

90. The process of claim 1, wherein the cytotoxic agent is DM1, the bifunctional crosslinking agent is SMCC, and the cell-binding agent is an anti-CD27L antibody.

91. The process of claim 1, wherein the cytotoxic agent is DM1, the bifunctional crosslinking agent is SMCC, and the cell-binding agent is trastuzumab.


SEQUENCE LISTING (4 sequences)

SEQ ID NO: 1 - protein, 450 aa, Artificial Sequence
  Synthetic Sequence
QVQLVQPGAEVVKPGASVKLSCKTSGYTFTSNWMHWVKQAPGQGLEWIGEIDPSDSYTNYNQNFQGKAKLTVDKSTSTAYMEVSSLRSDDTAVYYCARGSNPYYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 211 aa, Artificial Sequence
  Synthetic Sequence
EIVLTQSPAIMSASPGERVTMTCSASSGVNYMHWYQQKPGTSPRRWIYDTSKLASGVPARFSGSGSGTDYSLTISSMEPEDAATYYCHQRGSYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 115 aa, Artificial Sequence
  Synthetic Sequence
QVQVQESGPGLVAPSQTLSITCTVSGFSLTTSGVSWVRQPPGKGLEWLGVIWGDGSTNYHPSLKSRLSIKKDHSKSQVFLKLNSLTAADTATYYCAKGGYSLAHWGQGTLVTVSS

SEQ ID NO: 4 - protein, 108 aa, Artificial Sequence
  Sequence Listing
DIQMTQSPSSLSVSVGERVTITCRASENIRSNLAWYQQKPGKSPKLLVNVATNLADGVPSRFSGSGSGTDYSLKINSLQPEDFGTYYCQHYWGTTWTFGQGTKLEIKR